(19) United States
(12) Patent Application Publication
(10) Pub. No.: US 2007/0066916 A1
(43) Pub. Date: Mar. 22, 2007

(54) System and method for determining human emotion by analyzing eye properties

(75) Inventor: Jakob de Lemos, Copenhagen V (DK)

Correspondence Address: PILLSBURY WINTHROP SHAW PITTMAN, LLP, P.O. BOX 10500, MCLEAN, VA, 22102, US

(73) Assignee: iMotions Emotion Technology ApS, Copenhagen V (DK)

(21) Appl. No.: 11/522,476

(22) Filed: Sep. 18, 2006

Related U.S. Application Data
(60) Provisional application No. 60/717,268, filed on Sep. 16, 2005.

(51) Int. Cl. A61B 13/00

(52) U.S. Cl. 600/558

(57) ABSTRACT

The invention relates to a system and method for determining human emotion by analyzing a combination of eye properties of a user including, for example, pupil size, blink properties, eye position (or gaze) properties, or other properties. The system and method may be configured to measure the emotional impact of various stimuli presented to users by analyzing, among other data, the eye properties of the users while perceiving the stimuli. Measured eye properties may be used to distinguish between positive emotional responses (e.g., pleasant or “like”), neutral emotional responses, and negative emotional responses (e.g., unpleasant or “dislike”), as well as to determine the intensity of emotional responses.

### RELATED APPLICATION

This application claims priority from U.S. Provisional Patent Application No. 60/717,268, filed Sep. 16, 2005, and entitled “SYSTEM AND METHOD FOR DETERMINING HUMAN EMOTION BY MEASURING EYE PROPERTIES.” The contents of this provisional application is incorporated herein by reference.

### FIELD OF THE INVENTION

The invention relates generally to determining human emotion by analyzing eye properties including at least pupil size, blink properties, and eye position (or gaze) properties.

### BACKGROUND OF THE INVENTION

Systems and methods for tracking eye movements are generally known. In recent years, eye-tracking devices have made it possible for machines to automatically observe and record detailed eye movements. Some eye-tracking technology has been used, to some extent, to estimate a user's emotional state.

Despite recent advances in eye-tracking technology, many current systems suffer from various drawbacks. For instance, many existing systems which attempt to derive information about a user's emotions lack the ability to do so effectively, and/or accurately. Some fail to map results to a well-understood reference scheme or model including, among others, the “International Affective Picture System (IAPS) Technical Manual and Affective Ratings”, by Lang, P. J., Bradley, M. M., & Cuthbert, B. N., which is hereby incorporated herein by reference. As such, the results sometimes tend to be neither well understood nor widely applicable, in part due to the difficulty in deciphering them.

Moreover, existing systems do not appear to account for the importance of differentiating between emotional and rational processes in the brain when collecting data and/or reducing acquired data.

Additionally, some existing systems and methods fail to take into account relevant information that can improve the accuracy of a determination of a user's emotions. For example, some systems and methods fail to leverage the potential value in interpreting eye blinks as emotional indicators. Others fail to use other relevant information in determining emotions and/or confirming suspected emotions. Another shortcoming of prior approaches includes the failure to identify and take into account neutral emotional responses.

Many existing systems often use eye-tracking or other devices that are worn by or attached to the user. This invasive use of eye-tracking (and/or other) technology may itself impact a user's emotional state, thereby unnecessarily skewing the results.

These and other drawbacks exist with known eye-tracking systems and emotional detection methods.

### SUMMARY OF THE INVENTION

One aspect of the invention relates to solving these and other existing problems. According to one embodiment, the invention relates to a system and method for determining human emotion by analyzing a combination of eye properties of a user including, for example, pupil size, blink properties, eye position (or gaze) properties, or other properties. Measured eye properties, as described herein, may be used to distinguish between positive emotional responses (e.g., pleasant or “like”), neutral emotional responses, and negative emotional responses (e.g., unpleasant or “dislike”), as well as to determine the intensity of emotional responses.

As used herein, a “user” may, for example, refer to a respondent or a test subject, depending on whether the system and method of the invention are utilized in a clinical application (e.g., advertising or marketing studies or surveys, etc.) or a psychology study, respectively. In any particular data collection and/or analysis session, a user may comprise an active participant (e.g., responding to instructions, viewing and/or responding to various stimuli whether visual or otherwise, etc.) or a passive individual (e.g., unaware that data is being collected, not presented with stimuli, etc.). Additional nomenclature for a “user” may be used depending on the particular application of the system and method of the invention.

In one embodiment, the system and method of the invention may be configured to measure the emotional impact of various stimuli presented to users by analyzing, among other data, the eye properties of the users while perceiving the stimuli. The stimuli may comprise any real stimuli, or any analog or electronic stimuli that can be presented to users via known or subsequently developed technology. Any combination of stimuli relating to any one or more of a user's five senses (sight, sound, smell, taste, touch) may be presented.

The ability to measure the emotional impact of presented stimuli provides a better understanding of the emotional response to various types of content or other interaction scenarios. As such, the invention may be customized for use in any number of surveys, studies, interactive scenarios, or for other uses. As an exemplary illustration, advertisers may wish to present users with various advertising stimuli to better understand which types of advertising content elicit positive emotional responses. Similarly, stimulus packages may be customized for users by those involved in product design, computer game design, film analyses, media analyses, human computer interface development, e-learning application development, and home entertainment application development, as well as the development of security applications, safety applications, ergonomics, error prevention, or for medical applications concerning diagnosis and/or optimization studies. Stimulus packages may be customized for a variety of other fields or purposes.

According to an aspect of the invention, prior to acquiring data, a set-up and calibration process may occur. During set-up, if a user is to be presented with various stimuli during a data acquisition session, an administrator or other individual may either create a new stimulus package, or retrieve and/or modify an existing stimulus package. As recited above, any combination of stimuli relating to any one or more of a user's five senses may be utilized.

The set-up process may further comprise creating a user profile for a user including general user information (e.g., name, age, sex, etc.), general health information including information on any implanted medical devices that may introduce noise or otherwise negatively impact any sensor readings, eye-related information (e.g., use of contact lenses, use of glasses, any corrective laser eye surgery, diagnosis of or treatment for glaucoma or other condition), and information relating to general perceptions or feelings (e.g., likes or dis-likes) about any number of items including media, advertisements, etc. Other information may be included in a user profile.

In one implementation, calibration may comprise adjusting various sensors to an environment (and/or context), adjusting various sensors to the user within the environment, and determining a baseline emotional level for a user within the environment.

For example, when calibrating to an environment such as a room, vehicle, simulator, or other environment, ambient conditions (e.g., light, noise, temperature, etc.) may be measured so that either the ambient conditions, various sensors (e.g., cameras, microphones, scent sensors, etc.), or both may be adjusted accordingly to ensure that meaningful data (absent noise) can be acquired.

Additionally, one or more sensors may be adjusted to the user in the environment during calibration. For example, for the acquisition of eye-tracking data, a user may be positioned relative to an eye-tracking device such that the eye-tracking device has an unobstructed view of either the user's left eye, right eye, or both eyes. The eye-tracking device may not be physically attached to the user. In some implementations, the eye-tracking device may be visible to a user. In other implementations, the eye-tracking device may be positioned inconspicuously so that the user is unaware of the presence of the device. This may help to mitigate (if not eliminate) any instances of a user's emotional state being altered out of an awareness of the presence of the eye-tracking device. In yet another implementation, the eye-tracking device may be attached to or embedded in a display device, or other user interface. In still yet another implementation, the eye-tracking device may be worn by the user or attached to an object (e.g., a shopping cart) with which the user may interact in an environment during any number of various interaction scenarios.

The eye-tracking device may be calibrated to ensure that images of the user's eyes are clear, focused, and suitable for tracking eye properties of interest. Calibration may further comprise measuring and/or adjusting the level of ambient light present to ensure that any contraction or dilation of a user's pupils fall within what is considered to be a “neutral” or normal range. In one implementation, the calibration process may entail a user tracking, with his or her eyes, the movement of a visual indicator displayed on a display device positioned in front of the user. This process may be performed to determine where on the display device, as defined by position coordinates (e.g., x, y, z, or other coordinates), the user is looking. In this regard, a frame of reference for the user may be established.

A microphone (or other audio sensor) for speech or other audible input may also be calibrated (along with speech and/or voice recognition hardware and software) to ensure that a user's speech is acquired under optimal conditions. A galvanic skin response (GSR) feedback instrument used to measure skin conductivity from the fingers and/or palms may also be calibrated, along with a respiration rate belt sensor, EEG and EMG electrodes, or other sensors. Tactile sensors, scent sensors, and other sensors or known technology for monitoring various psycho-physiological conditions may be implemented. Other known or subsequently developed physiological and/or emotion detection techniques may be used with the eye-tracking data to enhance the emotion detection techniques disclosed herein.

In one implementation, various sensors may be simultaneously calibrated to an environment, and to the user within the environment. Other calibration protocols may be implemented.

According to an aspect of the invention, calibration may further comprise determining a user's emotional state (or level of consciousness) using any combination of known sensors (e.g., GSR feedback instrument, eye-tracking device, etc.) to generate baseline data for the user. Baseline data may be acquired for each sensor utilized.

In one implementation, calibration may further comprise adjusting a user's emotional state to ensure that the user is in as close to a desired emotional state (e.g., an emotionally neutral or other desired state) as possible prior to measurement, monitoring, or the presentation of any stimuli. In one implementation, various physiological data may be measured while presenting a user with stimuli known to elicit a positive (e.g., pleasant), neutral, or negative (e.g., unpleasant) response based on known emotional models. The stimuli may comprise visual stimuli or stimuli related to any of the body's other four senses. In one example, a soothing voice may address a user to place the user in a relaxed state of mind.

In one implementation, the measured physiological data may comprise eye properties. For example, a user may be presented with emotionally neutral stimuli until the blink rate pattern, pupil response, gaze movements, and/or other eye properties reach a desired level. In some embodiments, calibration may be performed once for a user, and calibration data may be stored with the user profile created for the user.

According to another aspect of the invention, after any desired initial set-up and/or calibration is complete, data may be collected for a user. This data collection may occur with or without the presentation of stimuli to the user. If a user is presented with stimuli, collected data may be synchronized with the presented stimuli. Collected data may include eye property data or other physiological data, environmental data, and/or other data.

According to one aspect of the invention, eye property data may be sampled at approximately 50 Hz., although other sampling frequencies may be used. Collected eye property data may include data relating to a user's pupil size, blink properties, eye position (or gaze) properties, or other eye properties. Data relating to facial expressions (e.g., movement of facial muscles) may also be collected. Collected pupil data may comprise, for example, pupil size, velocity of change (contraction or dilation), acceleration (which may be derived from velocity), or other pupil data. Collected blink data may comprise, for example, blink frequency, blink duration, blink potention, blink magnitude, or other blink data. Collected gaze data may comprise, for example, saccades, express saccades, nystagmus, or other gaze data. In some embodiments, as recited above, these properties may be measured in response to the user being presented with stimuli. The stimuli may comprise visual stimuli, non-visual stimuli, or a combination of both.

Although the system and method of the invention are described herein within the context of measuring the emotional impact of various stimuli presented to a user, it should be recognized that the various operations described herein may be performed absent the presentation of stimuli. As such, the description should not be viewed as limiting.

According to another aspect of the invention, collected data may be processed using one or more error detection and correction (data cleansing) techniques. Various error detection and correction techniques may be implemented for data collected from each of a number of sensors. With regard to collected eye property data, for example, error correction may include pupil light adjustment. Pupil size measurements, for instance, may be corrected to account for light sensitivity if not already accounted for during calibration, or even if accounted for during calibration. Error correction may further comprise blink error correction, gaze error correction, and outlier detection and removal. For those instances when a user is presented with stimuli, data that is unrelated to a certain stimulus (or stimuli) may be considered “outlier”. data and extracted. Other corrections may be performed.

According to an aspect of the invention, data processing may further comprise extracting (or determining) features of interest from data collected from each of a number of sensors. With regard to collected eye property data, for example, feature extraction may comprise processing pupil data, blink data, and gaze data for features of interest.

Processing pupil data may comprise, for example, determining pupil size (e.g., dilation or contraction) in response to a stimulus, determining the velocity of change (e.g., determining how fast a dilation or contraction occurs in response to a stimulus), as well as acceleration (which can be derived from velocity). Other pupil-related data including pupil base level and base distance may be determined as well as, for instance, minimum and maximum pupil sizes.

According to one aspect of the invention, processing blink data may comprise, for example, determining blink frequency, blink duration, blink potention, blink magnitude, or other blink data.

Processing gaze (or eye movement) data may comprise, for example, analyzing saccades, express saccades (e.g., saccades with a velocity greater than approximately 100 degrees per second), and nystagmus (rapid involuntary movements of the eye), or other data. Features of interest may include the velocity (deg/s) and direction of eye movements, fixation time (e.g., how long does the eye focus on one point), the location of the fixation in space (e.g., as defined by x,y,z or other coordinates), or other features.

According to another aspect of the invention, data processing may further comprise decoding emotional cues from collected and processed eye properties data (or other data) by applying one or more rules from an emotional reaction analysis engine (or module) to the processed data to determine one or more emotional components. Emotional components may include, for example, emotional valence, emotional arousal, emotion category (or name), and/or emotion type. Other components may be determined. Emotional valence may indicate whether a user's emotional response to a given stimulus is a positive emotional response (e.g., pleasant or “like”), negative emotional response (e.g., unpleasant or “dislike”), or neutral emotional response. Emotional arousal may comprise an indication of the intensity or “emotional strength” of the response using a predetermined scale.

In one implementation, the rules defined in the emotional reaction analysis engine (or module) may be based on established scientific findings regarding the study of various eye properties and their meanings. For instance, known relationships exist between a user's emotional valence and arousal, and eye properties such as pupil size, blink properties, and gaze.

Additional emotional components that may be determined from the processed data may include emotion category (or name), and/or emotion type. Emotion category (or name) may refer to any number of emotions described in any known or proprietary emotional model, while emotion type may indicate whether a user's emotional response to a given stimulus is instinctual or rational.

According to one aspect of the invention, a determination may be made as to whether a user has experienced an emotional response to a given stimulus. In one implementation, processed data may be compared to data collected and processed during calibration to see if any change from the emotionally neutral (or other) state measured (or achieved) during calibration has occurred. In another implementation, the detection of or determination that arousal has been experienced (based on the aforementioned feature decoding data processing) may indicate an emotional response. If no emotional response has been experienced, data collection may continue. If an emotional response has been detected, however, the emotional response may be evaluated.

When evaluating an emotional response, a determination may be made as to whether the emotional response comprises an instinctual or rational-based response. Within the very first second or seconds of perceiving a stimulus, or upon “first sight,” basic emotions (e.g., fear, anger, sadness, joy, disgust, interest, and surprise) may be observed as a result of activation of the limbic system and more particularly, the amygdala. These responses may be considered instinctual. Secondary emotions such as frustration, pride, and satisfaction, for instance, may result from the rational processing by the cortex within a longer time period (e.g., approximately one to five seconds) after perceiving a stimulus. While there is an active cooperation between the rational and the emotional processing of a given stimulus, it is advantageous to account for the importance of the instinctual response and its indication of human emotions. Very often, an initial period (e.g., a second) may be enough time for a human being to instinctually decide whether he or she likes or dislikes a given visual stimulus. This initial period is where the emotional impact really is expressed, before the cortex can return the first result of its processing and rational thinking takes over.

According to one embodiment, to determine whether a response is instinctual or rational, one or more rules from the emotional reaction analysis engine (or module) may be applied. If it is determined that the user's emotional response is an instinctual response, the data corresponding to the emotional response may be applied to an instinctual emotional impact model. However, if it is determined that the user's emotional response comprises a rational response, the data corresponding to the rational response may be applied to a rational emotional impact model.

According to an aspect of the invention, instinctual and rational emotional responses may be used in a variety of ways. One such use may comprise mapping the instinctual and rational emotional responses using 2-dimensional representations, 3-dimensional representations, graphical representations, or other representations. In some implementations, these maps may be displayed simultaneously and in synchronization with the stimuli that provoked them. In this regard, a valuable analysis tool is provided that may enable, for example, providers of content to view all or a portion of proposed content along with a graphical depiction of the emotional response it elicits from users.

Collected and processed data may be presented in a variety of manners. For example, according to one aspect of the invention, a gaze plot may be generated to highlight (or otherwise illustrate) those areas on a visual stimulus (e.g., a picture) that were the subject of most of a user's gaze fixation while the stimulus was being presented to the user. As recited above, processing gaze (or eye movement) data may comprise, among other things, determining fixation time (e.g., how long does the eye focus on one point) and the location of the fixation in space as defined by x,y,z or other coordinates. From this information, clusters of fixation points may be identified. In one implementation, a mask may be superimposed over a visual image or stimuli that was presented to a user. Once clusters of fixation points have been determined based on collected and processed gaze data that corresponds to the particular visual stimuli, those portions of the mask that correspond to the determined cluster of fixation points may be made transparent so as to reveal only those portions of the visual stimuli that a user focused on the most. Other data presentation techniques may be implemented.

In one implementation, results may be mapped to an adjective database which may aid in identifying adjectives for a resulting emotional matrix. This may assist in verbalizing or describing results in writing in one or more standardized (or industry-specific) vocabularies.

According to another aspect of the invention, statistical analyses may be performed on the results based on the emotional responses of several users or test subjects. Scan-path analysis, background variable analysis, and emotional evaluation analysis are each examples of the various types of statistical analyses that may be performed. Other types of statistical analyses may be performed.

According to an aspect of the invention, during human-machine interactive sessions, the interaction may be enhanced or content may be changed by accounting for user emotions relating to user input and/or other data. The methodology of the invention may be used in various artificial intelligence or knowledge-based systems applications to enhance or suppress desired human emotions. For example, emotions may be induced by selecting and presenting certain stimuli. Numerous other applications exist.

Depending on the application, emotion detection data (or results) may be published by, for example, incorporating data into a report, saving the data to a disk or other known storage device, transmitting the data over a network (e.g., the Internet), or otherwise presenting or utilizing the data. The data may also be used in any number of applications or in other manners, without limitation.

According to one aspect of the invention, a user may further be prompted to respond to verbal, textual, or other command-based inquiries about a given stimulus while (or after) the stimulus is presented to the user. In one example, a particular stimulus (e.g., a picture) may be displayed to a user. After a pre-determined time period, the user may be instructed to indicate whether he or she found the stimulus to be positive (e.g., pleasant), negative (e.g., unpleasant), or neutral, and/or the degree. Alternatively, the system may prompt the user to respond when the user has formed an opinion about a particular stimulus or stimuli. The time taken to form the opinion may be stored and used in a variety of ways. Users may register selections through any one of a variety of actions or gestures, for example, via a mouse-click in a pop-up window appearing on the display device, by verbally speaking the response into a microphone, or by other actions. Known speech and/or voice recognition technology may be implemented for those embodiments when verbal responses are desired. Any number and type of command-based inquiries may be utilized for requesting responses through any number of sensory input devices. In this regard, the measure of the emotional impact of a stimulus may be enhanced by including data regarding responses to command-based inquiries together with emotional data.

One advantage of the invention is that it differentiates between instinctual “pre-wired” emotional cognitive processing and “higher level” rational emotional cognitive processing, thus aiding in the elimination of “social learned behavioral “noise” in emotional impact testing.

Another advantage of the invention is that it provides “clean,” “first sight,” easy-to-understand, and easy-to-interpret data on a given stimulus.

These and other objects, features, and advantages of the invention will be apparent through the detailed description of the preferred embodiments and the drawings attached hereto. It is also to be understood that both the foregoing general description and the following detailed description are exemplary and not restrictive of the scope of the invention.

### BRIEF DESCRIPTION OF THE DRAWINGS

FIG. 1 provides a general overview of a method of determining human emotion by analyzing various eye properties of a user, according to an embodiment of the invention.

FIG. 2 illustrates a system for measuring the emotional impact of presented stimuli by analyzing eye properties, according to an embodiment of the invention.

FIG. 3 is an exemplary illustration of an operative embodiment of a computer, according to an embodiment of the invention.

FIG. 4 is an illustration of an exemplary operating environment, according to an embodiment of the invention.

FIG. 5 is a schematic representation of the various features and functionalities related to the collection and processing of eye property data, according to an embodiment of the invention.

FIG. 6 is an exemplary illustration of a block diagram depicting various emotional components, according to an embodiment of the invention.

FIG. 7 is an exemplary illustration of feature decoding operations, according to an embodiment of the invention.

FIGS. 8A-8D are graphical representations relating to a preliminary arousal operation, according to an embodiment of the invention.

FIG. 9 is exemplary illustration of a data table, according to an embodiment of the invention.

FIG. 10A-10H are graphical representations relating to a positive (e.g., pleasant) and negative (e.g., unpleasant) valence determination operation, according to an embodiment of the invention.

FIG. 11 illustrates an overview of instinctual versus rational emotions, according to an embodiment of the invention.

FIG. 12A is an exemplary illustration of a map of an emotional response, according to one embodiment of the invention.

FIG. 12B is an exemplary illustration of the Plutchiks emotional model.

FIG. 13 illustrates the display of maps of emotional responses together with the stimuli that provoked them, according to an embodiment of the invention.

### DETAILED DESCRIPTION OF THE PREFERRED EMBODIMENTS

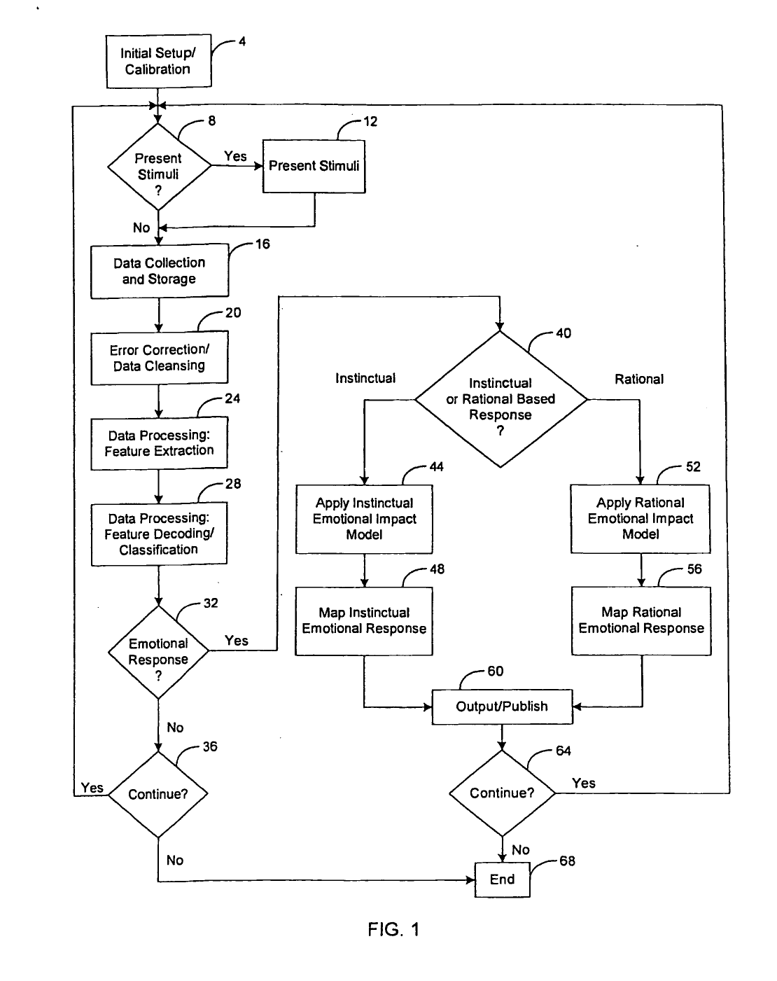

FIG. 1 provides a general overview of a method of determining human emotion by analyzing a combination of eye properties of a user, according to one embodiment of the invention. Although the method is described within the context of measuring the emotional impact of various stimuli presented to a user, it should be recognized that the various operations described herein may be performed absent the presentation of stimuli. For some uses, not all of the operations need be performed. For other uses, additional operations may be performed along with some or all of the operations shown in FIG. 1. In some implementations, one or more operations may be performed simultaneously. As such, the description should be viewed as exemplary, and not limiting.

Examples of various components that enable the operations illustrated in FIG. 1 will be described in greater detail below with reference to various ones of the figures. Not all of the components may be necessary. In some cases, additional components may be used in conjunction with some or all of the disclosed components. Various equivalents may also be used.

According to an aspect of the invention, prior to collecting data, a set-up and/or calibration process may occur in an operation 4. In one implementation, if a user is to be presented with stimuli during a data acquisition session, an administrator or other individual may either create a new stimulus package, or retrieve and/or modify an existing stimulus package. A stimulus package may, for example, comprise any combination of stimuli relating to any one or more of a user's five senses (sight, sound, smell, taste, touch). The stimuli may comprise any real stimuli, or any analog or electronic stimuli that can be presented to users via known technology. Stimuli may further comprise live scenarios such as, for instance, driving or riding in a vehicle, viewing a movie, etc. Various stimuli may also be combined to simulate various live scenarios in a simulator or other controlled environment.

Operation 4 may further comprise creating a user profile for a new user and/or modifying a profile for an existing user. A user profile may include general user information including, but not limited to, name, age, sex, or other general information. Eye-related information may also be included in a user profile, and may include information regarding any use of contact lenses or glasses, as well as any previous procedures such as corrective laser eye surgery, etc. Other eye-related information such as, for example, any diagnosis of (or treatment for) glaucoma or other conditions may also be provided. General health information may also be included in a user profile, and may include information on any implanted medical devices (e.g., a pacemaker) that may introduce noise or otherwise negatively impact any sensor readings during data collection. In addition, a user may also be prompted to provide or register general perceptions or feelings (e.g., likes, dis-likes) about any number of items including, for instance, visual media, advertisements, etc. Other information may be included in a user profile.

According to one aspect of the invention, in operation 4, various calibration protocols may be implemented including, for example, adjusting various sensors to an environment (and/or context), adjusting various sensors to a user within the environment, and determining a baseline emotional level for a user within the environment.

Adjusting or calibrating various sensors to a particular environment (and/or context) may comprise measuring ambient conditions or parameters (e.g., light intensity, background noise, temperature, etc.) in the environment, and if necessary, adjusting the ambient conditions, various sensors (e.g., cameras, microphones, scent sensors, tactile sensors, biophysical sensors, etc.), or both, to ensure that meaningful data can be acquired.

One or more sensors may also be adjusted (or calibrated) to a user in the environment during calibration. For the acquisition of eye-tracking data, for example, a user may be positioned (sitting, standing, or otherwise) relative to an eye-tracking device such that the eye-tracking device has an unobstructed view of either the user's left eye, right eye, or both eyes. In some instances, the eye-tracking device may not be physically attached to the user. In some implementations, the eye-tracking device may be positioned such that it is visible to a user. In other implementations, the eye-tracking device may be positioned inconspicuously in a manner that enables a user's eye properties to be tracked without the user being aware of the presence of the device. In this regard, any possibility that a user's emotional state may be altered out of an awareness of the presence of the eye-tracking device, whether consciously or subconsciously, may be minimized (if not eliminated). In another implementation, the eye-tracking device may be attached to or embedded in a display device.

In yet another implementation, however, the eye-tracking device may be worn by a user or attached to an object with which the user may interact in an environment during various interaction scenarios..

According to one aspect of the invention, the eye-tracking device may be calibrated to ensure that images of a single eye or of both eyes of a user are clear, focused, and suitable for tracking eye properties of interest. The level of ambient light present may also be measured and adjusted accordingly to ensure that any contraction or dilation of a user's pupils are within what is considered to be a “neutral” or normal range. In one implementation, during calibration, a user may be instructed to track, with his or her eyes, the movement of a visual indicator displayed on a display device positioned in front of the user to determine where on the display device, as defined by position coordinates (e.g., x, y, z, or other coordinates), the user is looking. In this regard, a frame of reference for the user may be established. In one implementation, the visual indicator may assume various shapes, sizes, or colors. The various attributes of the visual indicator may remain consistent during a calibration exercise, or vary. Other calibration methods may be used.

Additionally, in operation 4, any number of other sensors may calibrated for a user. For instance, a microphone (or other audio sensor) for speech or other audible input may be calibrated to ensure that a user's speech is acquired under optimal conditions. Speech and/or voice recognition hardware and software may also be calibrated as needed. A respiration rate belt sensor, EEG and EMG electrodes, and a galvanic skin response (GSR) feedback instrument used to measure skin conductivity from the fingers and/or palms may also be calibrated, along with tactile sensors, scent sensors, or any other sensors or known technology for monitoring various psycho-physiological conditions. Other known or subsequently developed physiological and/or emotion detection techniques (and sensors) may be used with the eye-tracking data to enhance the emotion detection techniques disclosed herein.

In one implementation, various sensors may be simultaneously calibrated to an environment, and to the user within the environment. Other calibration protocols may be implemented.

According to one aspect of the invention, in operation 4, calibration may further comprise determining a user's current emotional state (or level of consciousness) using any combination of known sensors to generate baseline data for the user. Baseline data may be acquired for each sensor utilized.

In one implementation, a user's emotional level may also be adjusted, in operation 4, to ensure that a user is in as close to a desired emotional state (e.g., an emotionally neutral or other desired state) as possible prior to measurement, monitoring, or the presentation of any stimuli. For example, various physiological data may be measured while the user is presented with images or other stimuli known to elicit a positive (e.g., pleasant), neutral, or negative (e.g., unpleasant) response based on known emotional models. In one example, if measuring eye properties, a user may be presented with emotionally neutral stimuli until the blink rate pattern, pupil response, saccadic movements, and/or other eye properties reach a desired level. Any single stimulus or combination of stimuli related to any of the body's five senses may be presented to a user. For example, in one implementation, a soothing voice may address a user to place the user in a relaxed state of mind. The soothing voice may (or may not) be accompanied by pleasant visual or other stimuli.

According to some embodiments of the invention, calibration may be performed once for a user. Calibration data for each user may be stored either together with (or separate from) a user profile created for the user.

According to an aspect of the invention, once any desired set-up and/or calibration is complete, data may be collected for a user. This data collection may occur with or without the presentation of stimuli to the user. For example, in an operation 8, a determination may be made as to whether stimuli will be presented to a user during data collection. If a determination is made that data relating to the emotional impact of presented stimuli on the user is desired, stimuli may be presented to the user in operation 12 and data may be collected in an operation 16 (described below). By contrast, if the determination is made in operation 8 that stimuli will not be presented to the user, data collection may proceed in operation 16.

In operation 16, data may be collected for a user. Collected data may comprise eye property data or other physiological data, environmental data, and/or other data. If a user is presented with stimuli (operation 12), collected data may be synchronized with the presented stimuli.

According to one aspect of the invention, eye property data may be sampled at approximately 50 Hz. or at another suitable sampling rate. Collected eye property data may include data relating to a user's pupil size, blink properties, eye position (or gaze) properties, or other eye properties. Collected pupil data may comprise pupil size, velocity of change (contraction or dilation), acceleration (which may be derived from velocity), or other pupil data. Collected blink data may include, for example, blink frequency, blink duration, blink potention, blink magnitude, or other blink data. Collected gaze data may comprise, for example, saccades, express saccades, nystagmus, or other gaze data. Data relating to the movement of facial muscles (or facial expressions in general) may also be collected.

According to an aspect of the invention, the data collected in operation 16 may be processed using one or more error detection and correction (data cleansing) techniques in an operation 20. Various error detection and correction techniques may be implemented for data collected from each of the sensors used during data collection. For example, for collected eye property data, error correction may include pupil light adjustment. Pupil size measurements, for instance, may be corrected to account for light sensitivity if not already accounted for during calibration, or even if accounted for during calibration. Error correction may further comprise blink error correction, gaze error correction, and outlier detection and removal. For those instance when a user is presented with stimuli, data that is unrelated to a certain stimulus (or stimuli) may be considered “outlier” data and extracted. Other corrections may be performed.

In an operation 24, data processing may further comprise extracting (or determining) features of interest from data collected by a number of sensors. With regard to collected eye property data, feature extraction may comprise processing pupil data, blink data, and gaze data for features of interest.

Processing pupil data, in operation 24, may comprise, for example, determining pupil size (e.g., dilation or contraction) in response to a stimulus. Processing pupil data may further comprise determining the velocity of change or how fast a dilation or contraction occurs in response to a stimulus, as well as acceleration which can be derived from velocity. Other pupil-related data including pupil base level and base distance may be determined as well as, for instance, minimum and maximum pupil sizes.

Processing blink data, in operation 24, may comprise, for example, determining blink frequency, blink duration, blink potention, blink magnitude, or other blink data. Blink frequency measurement may include determining the timeframe between sudden blink activity.

Blink duration (in, for example, milliseconds) may also be processed to differentiate attentional blinks from physiological blinks. Five blink patterns may be differentiated based on their duration. Neutral blinks may be classified as those which correspond to the blinks measured during calibration. Long blink intervals may indicate increased attention, while short blinks indicate that the user may be searching for information. Very short blink intervals may indicate confusion, while half-blinks may serve as an indication of a heightened sense of alert. Blink velocity refers to how fast the amount of eyeball visibility is changing, while the magnitude of a blink refers to how much of the eyeball is visible while blinking.

Processing gaze (or eye movement data), in operation 24, may comprise, for example, analyzing saccades, express saccades (e.g., saccades with a velocity greater than approximately 100 degrees per second), and nystagmus (rapid involuntary movements of the eye), or other data. Features of interest may include the velocity (deg/s) and direction of eye movements, fixation time (e.g., how long does the eye focus on one point), the location of the fixation in space (e.g., as defined by x,y,z or other coordinates), or other features including return to fixation areas, relevance, vergence for depth evaluation, and scan activity.

According to an aspect of the invention, in an operation 28, data processing may comprise decoding emotional cues from eye properties data collected and processed (in operations 16, 20, and 24) by applying one or more rules from an emotional reaction analysis engine (or module) to the processed data to determine one or more emotional components. Emotional components may include, for example, emotional valence, emotional arousal, emotion category (or name), and/or emotion type. Other components may be determined.

Emotional valence may be used to indicate whether a user's emotional response to a given stimulus is a positive emotional response (e.g., pleasant or “like”), a negative emotional response (e.g., unpleasant or “dislike”), or neutral emotional response.

Emotional arousal may comprise an indication of the intensity or “emotional strength” of the response using a predetermined scale. For example, in one implementation, this value may be quantified on a negative to positive scale, with zero indicating a neutral response. Other measurement scales may be implemented.

According to one implmentation, the rules defined in the emotional reaction analysis engine (or module) may be based on established scientific findings regarding the study of various eye properties and their meanings. For example, a relationship exists between pupil size and arousal. Additionally, there is a relationship between a user's emotional valence and pupil dilation. An unpleasant or negative reaction, for example, may cause the pupil to dilate larger than a pleasant or neutral reaction.

Blink properties also aid in defining a user's emotional valence and arousal. With regard to valence, an unpleasant response may be manifested in quick, half-closed blinks. A pleasant, positive response, by contrast, may result in long, closed blinks. Negative or undesirable stimuli may result in frequent surprise blinks, while pleasant or positive stimuli may not result in significant surprise blinks. Emotional arousal may be evaluated, for example, by considering the velocity of blinks. Quicker blinks may occur when there is a stronger emotional reaction.

Eye position and movement may also be used to deduce emotional cues. By measuring how long a user fixates on a particular stimulus or portion of a stimulus, a determination can be made as to whether the user's response is positive (e.g., pleasant) or negative (e.g., unpleasant). For example, a user staring at a particular stimulus may indicate a positive (or pleasant) reaction to the stimulus, while a negative (or unpleasant) reaction may be inferred if the user quickly looks away from a stimulus.

Additional emotional components that may be determined from the processed data may include emotion category (or name), and/or emotion type.

Emotion category (or name) may refer to any number of emotions (e.g., joy, sadness, anticipation, surprise, trust, disgust, anger, fear,. etc.) described in any known or proprietary emotional model. Emotion type may indicate whether a user's emotional response to a given stimulus is instinctual or rational.

According to one aspect of the invention, a determination may be made, in an operation 32, as to whether a user has experienced an emotional response to a given stimulus. In one implementation, processed data may be compared to data collected and processed during calibration to see if any change from the emotionally neutral (or other) state measured (or achieved) during calibration has occurred. In another implementation, the detection of or determination that arousal has been experienced (based on the aforementioned feature decoding data processing) may indicate an emotional response.

If a determination is made in operation 32 that no emotional response has been experienced, a determination may be made in an operation 36 as to whether to continue data collection. If additional data collection is desired, processing may continue with operation 8 (described above). If no additional data collection is desired, processing may end in an operation 68.

If a determination is made in operation 32, however, that an emotional response has been detected, the emotional response may be evaluated. In an operation 40, for example, a determination may be made as to whether the emotional response comprises an instinctual or rational-based response. Within the very first second or seconds of perceiving a stimulus, or upon “first sight,” basic “instinctual” emotions (e.g., fear, anger, sadness, joy, disgust, interest, and surprise) may be observed as a result of activation of the limbic system and more particularly, the amygdala. Secondary emotions such as frustration, pride, and satisfaction, for instance, may result from the rational processing of the cortex within a time frame of approximately one to five seconds after perceiving a stimulus. Accordingly, although there is an active cooperation between the rational and the emotional processing of a given stimulus, it is advantageous to account for the importance of the “first sight” and its indication of human emotions.

In this regard, collected data may be synchronized with presented stimuli, so that it can be determined which portion of collected data corresponds to which presented stimulus. For example, if a first stimulus (e.g., a first visual image) is displayed to a user for a predetermined time period, the corresponding duration of collected data may include metadata (or some other data record) indicating that that duration of collected data corresponds to the eye properties resulting from the user's reaction to the first image. The first second or so of the predetermined duration may, in some implementations, be analyzed in depth. Very often, an initial period (e.g., a second) may be enough time for a human being to instinctually decide whether he or she likes or dislikes a given stimulus. This initial period is where the emotional impact really is expressed, before the cortex can return the first result of its processing and rational thinking takes over.

According to an aspect of the invention, in operation 40, one or more rules from the emotional reaction analysis engine (or module) may be applied to determine whether the response is instinctual or rational. For example, sudden pupil dilation, smaller blink sizes, and/or other properties may indicate an instinctual response, while a peak in dilation and larger blink sizes may indicate a rational reaction. Other predefined rules may be applied.

If a determination is made, in operation 40, that the user's emotional response is an instinctual response, the data corresponding to the emotional response may be applied to an instinctual emotional impact model in an operation 44.

By contrast, if it is determined in operation 40, that the user's emotional response comprises a rational response, the data corresponding to the rational response may be applied to a rational emotional impact model in an operation 52.

Some examples of known emotional models that may be utilized by the system and method described herein include the Ekmans, Plutchiks, and Izards models. Ekmans emotions are related to facial expressions such as anger, disgust, fear, joy, sadness, and surprise. The Plutchiks model expands Ekmans basic emotions to acceptance, anger, anticipation, disgust, joy, fear, sadness, and surprise. The Izards model differentiates between anger, contempt, disgust, fear, guilt, interest, joy, shame, and surprise.

In one implementation of the invention, in operations 48 and 56, instinctual and rational emotional responses, respectively, may be mapped in a variety of ways (e.g., 2 or 3-dimensional representations, graphical representations, or other representations). In some implementations, these maps may be displayed simultaneously and in synchronization with the stimuli that provoked them. In this regard, a valuable analysis tool is provided that may enable, for example, providers of content to view all or a portion of proposed content along with a graphical depiction of the emotional response it elicits from users.

Depending on the application, emotion detection data (or results) may be published or otherwise output in an operation 60. Publication may comprise, for example, incorporating data into a report, saving the data to a disk or other known storage device, transmitting the data over a network (e.g., the Internet), or otherwise presenting or utilizing the data. The data may be used in any number of applications or in other manners, without limitation.

Although not shown in the general overview of the method depicted in FIG. 1, one embodiment of the invention may further comprise prompting a user to respond to command-based inquiries about a given stimulus while (or after) the stimulus is presented to the user. The command-based inquiries may be verbal, textual, or otherwise. In one implementation, for instance, a particular stimulus (e.g., a picture) may be displayed to a user. After a pre-determined time period, the user may be instructed to select whether he or she found the stimulus to be positive (e.g., pleasant), negative (e.g., unpleasant), or neutral and/or the degree.

A user may alternatively be prompted, in some implementations, to respond when he or she has formed an opinion about a particular stimulus or stimuli. The time taken to form the opinion may be stored or used in a variety of ways. The user may register selections through any one of a variety of actions or gestures, for example, via a mouse-click in a pop-up window appearing on the display device, verbally by speaking the response into a microphone, or by other actions. Known speech and/or voice recognition technology may be implemented for those embodiments when verbal responses are desired. Any number and type of command-based inquiries may be utilized for requesting responses through any number of sensory input devices. In this regard, the measure of the emotional impact of a stimulus may be enhanced by including data regarding responses to command-based inquiries together with emotional data. Various additional embodiments are described in detail below.

Having provided an overview of a method of determining human emotion by analyzing a combination of eye properties of a user, the various components which enable the operations illustrated in FIG. 1 will now be described.

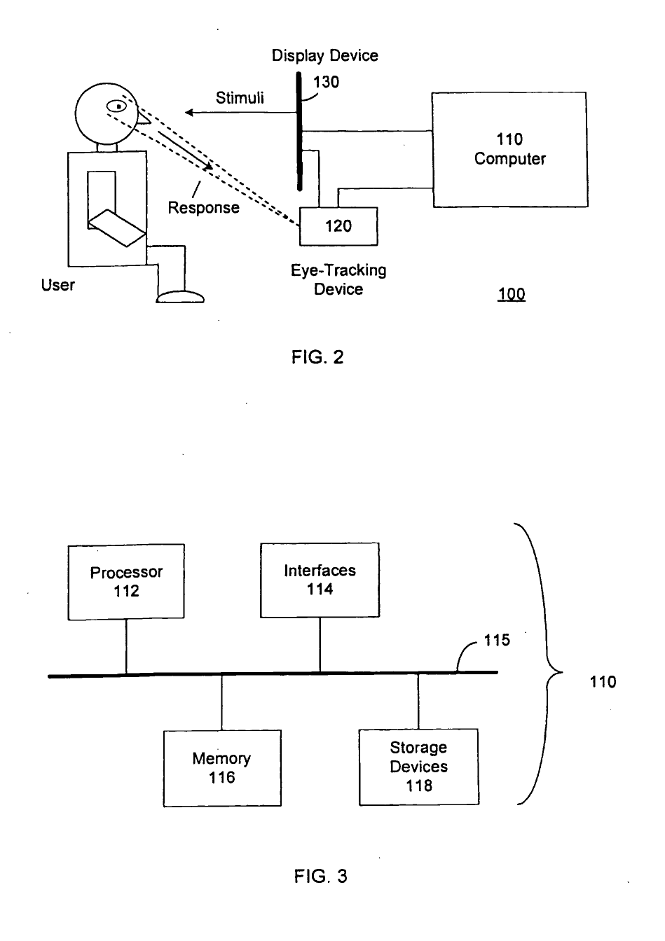

According to an embodiment of the invention illustrated in FIG. 2, a system 100 is provided for determining human emotion by analyzing a combination of eye properties of a user. In one embodiment, system 100 may be configured to measure the emotional impact of stimuli presented to a user by analyzing eye properties of the user. System 100 may comprise a computer 110, eye-tracking device 120, and a display device 130, each of which may be in operative communication with one another.

Computer 110 may comprise a personal computer, portable computer (e.g., laptop computer), processor, or other device. As shown in FIG. 3, computer 110 may comprise a processor 112, interfaces 114, memory 116, and storage devices 118 which are electrically coupled via bus 115. Memory 116 may comprise random access memory (RAM), read only memory (ROM), or other memory. Memory 116 may store computer-executable instructions to be executed by processor 112 as well as data which may be manipulated by processor 112. Storage devices 118 may comprise floppy disks, hard disks, optical disks, tapes, or other known storage devices for storing computer-executable instructions and/or data.

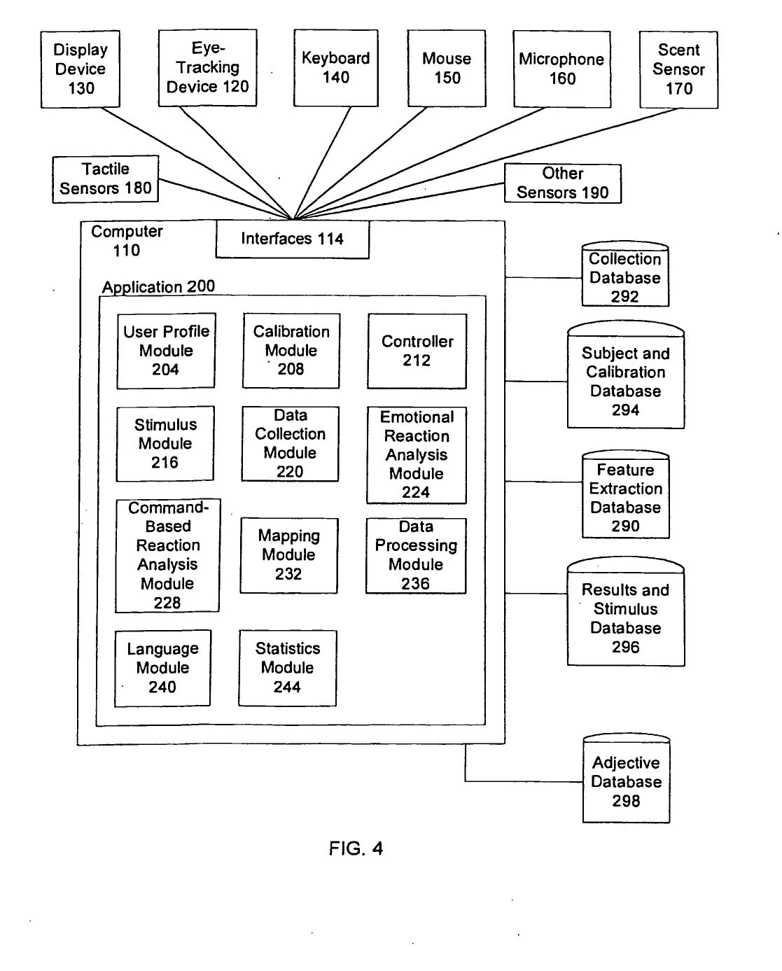

With reference to FIG. 4, interfaces 114 may comprise an interface to display device 130 that may be used to present stimuli to users. Interface 114 may further comprise interfaces to peripheral devices used to acquire sensory input information from users including eye tracking device 120, keyboard 140, mouse 150, one or more microphones 160, one or more scent sensors 170, one or more tactile sensors 180, and other sensors 190. Other sensors 190 may include, but are not limited to, a respiration belt sensor, EEG electrodes, EMG electrodes, and a galvanic skin response (GSR) feedback instrument used to measure skin conductivity from the fingers and/or palms. Other known or subsequently developed physiological and/or emotion detection sensors may be used. Interfaces 114 may further comprise interfaces to other devices such as a printer, a display monitor (separate from display device 130), external disk drives or databases.

According to an aspect of the invention, eye-tracking device 120 may comprise a camera or other known eye-tracking device that records (or tracks) various eye properties of a user. Examples of eye properties that may be tracked by eye-tracking device 120, as described in greater detail below, may include pupil size, blink properties, eye position (or gaze) properties, or other properties. Eye-tracking device 120 may comprise a non-intrusive, non-wearable device that is selected to affect users as little as possible. In some implementations, eye-tracking device 120 may be positioned such that it is visible to a user. In other implementations, eye-tracking device 120 may be positioned inconspicuously in a manner that enables a user's eye properties to be tracked without the user being aware of the presence of the device.

According to one aspect of the invention, eye-tracking device 120 may not be physically attached to a user. In this regard, any possibility of a user altering his or her responses (to stimuli) out of an awareness of the presence of eye-tracking device 120, whether consciously or subconsciously, may be minimized (if not eliminated).

Eye-tracking device 120 may also be attached to or embedded in display device 130 (e.g., similar to a camera in a mobile phone). In one implementation, eye-tracking device 120 and/or display device 130 may comprise the “Tobii 1750 eye-tracker” commercially available from Tobii Technology AB. Other commercially available eye-tracking devices and/or technology may be used in place of, or integrated with, the various components described herein.

According to another implementation, eye-tracking device 120 may be worn by a user or attached to an object with which the user may interact in an environment during various interaction scenarios.

According to an aspect of the invention, display device 130 may comprise a monitor or other display device for presenting visual (or other) stimuli to a user via a graphical user interface (GUI). As described in greater detail below, visual stimuli may include, for example, pictures, artwork, charts, graphs, movies, multimedia presentations, interactive content (e.g., video games) or simulations, or other visual stimuli.

In one implementation, display device 130 may be provided in addition to a display monitor associated with computer 110. In an alternative implementation, display device 130 may comprise the display monitor associated with computer 110.

As illustrated in FIG. 4, computer 110 may run an application 200 comprising one or modules for determining human emotion by analyzing data collected on a user from various sensors. Application 200 may be further configured for presenting stimuli to a user, and for measuring the emotional impact of the presented stimuli. Application 200 may comprise a user profile module 204, calibration module 208, controller 212, stimulus module 216, data collection module 220, emotional reaction analysis module 224, command-based reaction analysis module 228, mapping module 232, data processing module 236, language module 240, statistics module 244, and other modules, each of which may implement the various features and functions (as described herein). One or more of the modules comprising application 200 may be combined. For some purposes, not all modules may be necessary.

The various features and functions of application 200 may be accessed and navigated by a user, an administrator, or other individuals via a GUI displayed on either or both of display device 130 or a display monitor associated with computer 110. The features and functions of application 200 may also be controlled by another computer or processor.

In various embodiments, as would be appreciated, the functionalities described herein may be implemented in various combinations of hardware and/or firmware, in addition to, or instead of, software.

According to one embodiment, computer 110 may host application 200. In an alternative embodiment, not illustrated, application 200 may be hosted by a server. Computer 110 may access application 200 on the server over a network (e.g., the Internet, an intranet, etc.) via any number of known communications links. In this embodiment, the invention may be implemented in software stored as executable instructions on both the server and computer 110. Other implementations and configurations may exist depending on the particular type of client/server architecture implemented.

Various other system configurations may be used. As such, the description should be viewed as exemplary, and not limiting.

In one implementation, an administrator or operator may be present (in addition to a user) to control the various features and functionality of application 200 during either or both of an initial set-up/calibration process and a data acquisition session.

In an alternative implementation, a user may control application 200 directly, without assistance or guidance, to self-administer either or both of the initial set-up/calibration process and a data acquisition session. In this regard, the absence of another individual may help to ensure that a user does not alter his or her emotional state out of nervousness or self-awareness which may be attributed to the presence of another individual. In this implementation, computer 110 may be positioned in front of (or close enough to) the user to enable the user to access and control application 200, and display device 130 may comprise the display monitor associated with computer 110. As such, a user may navigate the various modules of application 200 via a GUI associated with application 200 that may be displayed on display device 130. Other configurations may be implemented.

According to one aspect of the invention, if a user is to be presented with stimuli during a data acquisition session, a user, administrator, or other individual may either create a new stimulus package, or retrieve and/or modify an existing stimulus package as part of the initial set-up. The creation and modification, and presentation of various stimulus packages may be enabled by stimulus module 216 of application 200 using a GUI associated with the application. Stimulus packages may be stored in a results and stimulus database 296.

According to one aspect of the invention, a stimulus package may comprise any combination of stimuli relating to any one or more of a user's five senses (sight, sound, smell, taste, touch). The stimuli may comprise any real stimuli, or any analog or electronic stimuli that can be presented to users via known technology. Examples of visual stimuli, for instance, may comprise pictures, artwork, charts, graphs, movies, multimedia presentations, interactive content (e.g., video games), or other visual stimuli. Stimuli may further comprise live scenarios such as, for instance, driving or riding in a vehicle, viewing a movie, etc. Various stimuli may also be combined to simulate various live scenarios in a simulator or other controlled environment.

The stimulus module 216 may enable various stimulus packages to be selected for presentation to users depending on the desire to understand emotional response to various types of content. For example, advertisers may present a user with various advertising stimuli to better understand to which type of advertising content the user may react positively (e.g., like), negatively (e.g., dislike), or neutrally. Similarly, the stimulus module may allow stimulus packages to be customized for those involved in product design, computer game design, film analyses, media analyses, human computer interface development, e-learning application development, and home entertainment application development, as well as the development of security applications, safety applications, ergonomics, error prevention, or for medical applications concerning diagnosis and/or optimization studies. Stimulus packages may be customized for a variety of other fields or purposes.

According to one aspect of the invention, during initial set-up, user profile module 204 (of application 200) may prompt entry of information about a user (via the GUI associated with application 200) to create a user profile for a new user. User profile module 204 may also enable profiles for existing users to be modified as needed. In addition to name, age, sex, and other general information, a user may be prompted to enter information regarding any use of contact lenses or glasses, as well as any previous procedures such as, for example, corrective laser eye surgery, etc. Other eye-related information including any diagnosis of (or treatment for) glaucoma or other conditions may be included. A user profile may also include general health information, including information on any implanted medical devices (e.g., a pacemaker) that may introduce noise or otherwise negatively impact any sensor readings during data collection. A user may further be prompted to provide or register general perceptions or feelings (e.g., likes, dis-likes) about any number of items including, for instance, visual media, advertisements, etc. Other information may be included in a user profile. Any of the foregoing information may be inputted by either a user or an administrator, if present. In one embodiment, user profiles may be stored in subject and calibration database 294.

According to one aspect of the invention, various calibration protocols may be implemented including, for example, adjusting various sensors to an environment (and/or context), adjusting various sensors to a user within the environment, and determining a baseline emotional level for a user within the environment.

Adjusting or calibrating various sensors to a particular environment (and/or context) may comprise measuring ambient conditions or parameters (e.g., light intensity, background noise, temperature, etc.) in the environment, and if necessary, adjusting the ambient conditions, various sensors (e.g., eye-tracking device 120, microphone 160, scent sensors 170, tactile sensors 180, and/or other sensors 190), or both, to ensure that meaningful data can be acquired.

According to one aspect of the invention, one or more sensors may be adjusted or calibrated to a user in the environment during calibration. For the collection of eye-tracking data, for example, a user may be positioned (sitting, standing, or otherwise) such that eye-tracking device 120 has an unobstructed view of either the user's left eye, right eye, or both eyes. In one implementation, controller 212 may be utilized to calibrate eye-tracking device 120 to ensure that images of a single eye or of both eyes are clear, focused, and suitable for tracking eye properties of interest. The level of ambient light present may also be measured and adjusted accordingly to ensure that a user's pupils are neither dilated nor contracted outside of what is considered to be a “neutral” or normal range. Controller 212 may be a software module including for example a hardware driver, that enables a hardware device to be controlled and calibrated.

Calibration module 208 may enable a calibration process wherein a user is asked to track, with his or her eyes, the movement of a visual indicator displayed on display device 130 to determine where on display device 130, as defined by position coordinates (e.g., x, y, z, or other coordinates), the user is looking. In this regard, a frame of reference for a user may be established. The visual indicator may assume various shapes, sizes, or colors. The various attributes of the visual indicator may remain consistent during a calibration exercise, or vary. Other calibration methods may be used.

Calibration module 208 and/or controller 212 may enable any number of other sensors to be calibrated for a user. For example, one or more microphones 160 (or other audio sensors) for speech or other audible input may be calibrated to ensure that a user's speech is acquired under optimal conditions. Speech and/or voice recognition hardware and software may also be calibrated as needed. Scent sensors 170, tactile sensors 180, and other sensors 190 including a respiration rate belt sensor, EEG and EMG electrodes, and a GSR feedback instrument may also be calibrated, as may additional sensors.

In one implementation, various sensors may be simultaneously calibrated to an environment, and to the user within the environment. Other calibration protocols may be implemented.

Calibration may further comprise determining a user's current emotional state (or level of consciousness) using any combination of known sensors to generate baseline data for the user. Baseline data may be acquired for each sensor utilized.

In one implementation, a user's emotional level may also be adjusted to ensure that a user is in as close to a desired emotional state (e.g., an emotionally neutral or other desired state) as possible prior to measurement, monitoring, or the presentation of any stimuli. For example, various physiological data may be measured by presenting a user with images or other stimuli known to elicit a positive (e.g., pleasant), neutral, or negative (e.g., unpleasant) response based on known emotional models.

In one example, if measuring eye properties, a user may be shown emotionally neutral stimuli until the blink rate pattern, pupil response, saccadic movements, and/or other eye properties reach a desired level. Any single stimulus or combination of stimuli related to any of the body's five senses may be presented to a user. For example, in one implementation, a soothing voice may address a user to place the user in a relaxed state of mind. The soothing voice may (or may not) be accompanied by pleasant visual or other stimuli. The presentation of calibration stimuli may be enabled by either one or both of calibration module 208 or stimulus module 216.

According to some embodiments of the invention, calibration may be performed once for a user. Calibration data for each user may be stored in subject and calibration database 294 together with (or separate from) their user profile.

According to an aspect of the invention, once any desired set-up and/or calibration is complete, data may be collected and processed for a user. Data collection module 220 may receive raw data acquired by eye-tracking device 120, or other sensory input devices. Collected data may comprise eye property data or other physiological data, environmental data (about the testing environment), and/or other data. The raw data may be stored in collection database 292, or in another suitable data repository. Data collection may occur with or without the presentation of stimuli to a user.

In one implementation, if stimuli is presented to a user, it may be presented using any number of output devices. For example, visual stimuli may be presented to a user via display device 130. Stimulus module 216 and data collection module 220 may be synchronized so that collected data may be synchronized with the presented stimuli.

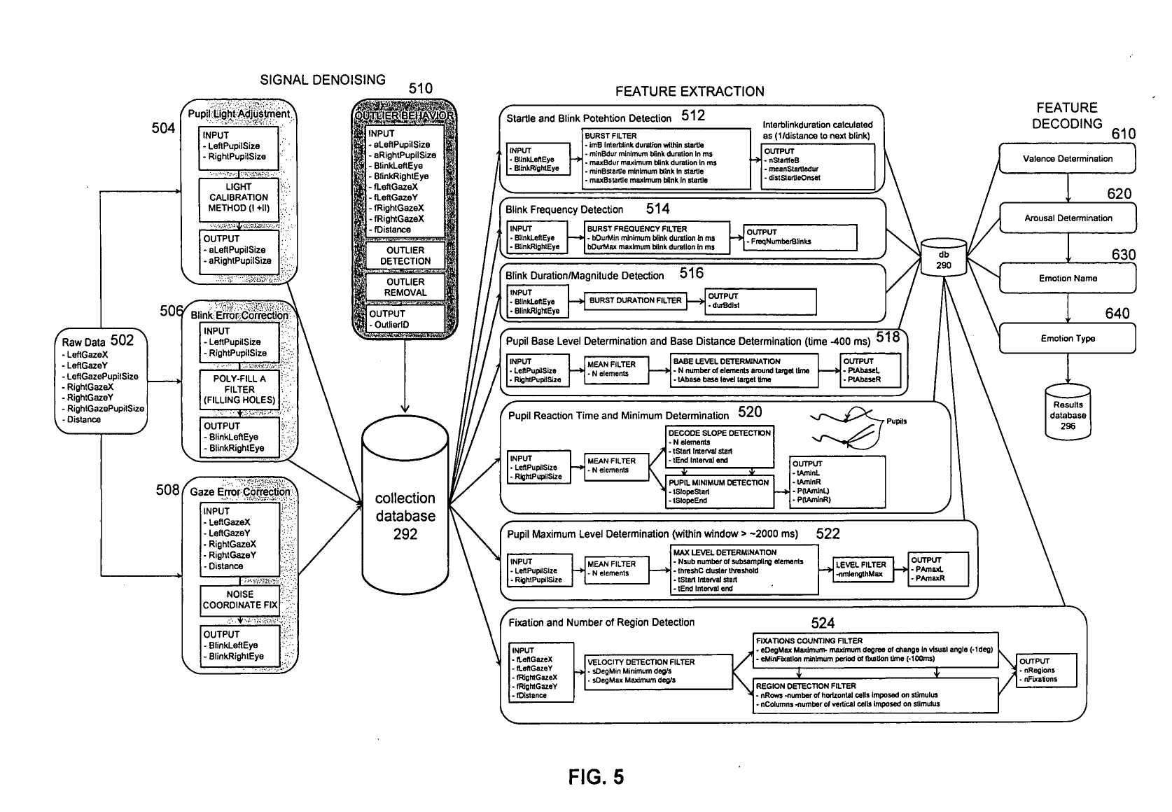

FIG. 5 is a schematic representation of the various features and functionalities enabled by application 200 (FIG. 4), particularly as they relate to the collection and processing of eye property data, according to one implementation. The features and functionalities depicted in FIG. 5 are explained herein.

According to one aspect of the invention, data collection module 220, may sample eye property data at approximately 50 Hz., although other suitable sampling rates may be used. The data collection module 220 may further collect eye property data including data relating to a user's pupil size, blink properties, eye position (or gaze) properties, or other eye properties. Collected pupil data may comprise pupil size, velocity of change (contraction or dilation), acceleration (which may be derived from velocity), or other pupil data. Collected blink data may include, for example, blink frequency, blink duration, blink potention, blink magnitude, or other blink data. Collected gaze data may comprise, for example, saccades, express saccades, nystagmus, or other gaze data. Data relating to the movement of facial muscles (or facial expressions in general) may also be collected. These eye properties may be used to determine a user's emotional reaction to one or more stimuli, as described in greater detail below.

According to an aspect of the invention, collected data may be processed (e.g., by data processing module 236) using one or more signal denoising or error detection and correction (data cleansing) techniques. Various error detection and correction techniques may be implemented for data collected from each of the sensors used during data collection.

For example, and as shown in FIG. 5, for collected eye property data including for example, raw data 502, error correction may include pupil light adjustment 504. Pupil size measurements, for instance, may be corrected to account for light sensitivity if not already accounted for during calibration, or even if accounted for during calibration. Error correction may further comprise blink error correction 506, gaze error correction 508, and outlier detection and removal 510. For those instances when a user is presented with stimuli, data that is unrelated to a certain stimulus (or stimuli) may be considered “outlier” data and extracted. Other corrections may be performed. In one implementation, cleansed data may also be stored in collection database 292, or in any other suitable data repository.

According to one aspect of the invention, data processing module 236 may further process collected and/or “cleansed” data from collection database 292 to extract (or determine) features of interest from collected data. With regard to collected eye property data, and as depicted in FIG. 5, feature extraction may comprise processing pupil data, blink data, and gaze data to determine features of interest. In one implementation various filters may be applied to input data to enable feature extraction.

Processing pupil data may comprise, for example, determining pupil size (e.g., dilation or contraction) in response to a stimulus. Pupil size can range from approximately 1.5 mm to more than 9 mm. Processing pupil data may further comprise determining the velocity of change or how fast a dilation or contraction occurs in response to a stimulus, as well as acceleration which can be derived from velocity. Other pupil-related data including pupil base level and base distance 518 may be determined as well as, for instance, minimum and maximum pupil sizes (520, 522).

Processing blink data may comprise, for example, determining blink potention 512, blink frequency 514, blink duration and blink magnitude 516, or other blink data. Blink frequency measurement may include determining the timeframe between sudden blink activity.

Blink duration (in, for example, milliseconds) may also be processed to differentiate attentional blinks from physiological blinks. Five blink patterns may be differentiated based on their duration. Neutral blinks may be classified as those which correspond to the blinks measured during calibration. Long blink intervals may indicate increased attention, while short blinks indicate that the user may be searching for information. Very short blink intervals may indicate confusion, while half-blinks may serve as an indication of a heightened sense of alert. Blink velocity refers to how fast the amount of eyeball visibility is changing while the magnitude of a blink refers to how much of the eyeball is visible while blinking.

Processing gaze (or eye movement data) 524 may comprise, for example, analyzing saccades, express saccades (e.g., saccades with a velocity greater than approximately 100 degrees per second), and nystagmus (rapid involuntary movements of the eye), or other data. Features of interest may include the velocity (deg/s) and direction of eye movements, fixation time (e.g., how long does the eye focus on one point), the location of the fixation in space (e.g., as defined by x,y,z or other coordinates), or other features including return to fixation areas, relevance, vergence for depth evaluation, and scan activity.

Extracted feature data may be stored in feature extraction database 290, or in any other suitable data repository.

According to another aspect of the invention, data processing module 236 may decode emotional cues from extracted feature data (stored in feature extraction database 290) by applying one or more rules from an emotional reaction analysis module 224 to the data to determine one or more emotional components including, emotional valence 610, emotional arousal 620, emotion category (or name) 630, and/or emotion type 640. As shown in FIG. 5, and described in greater detail below, the results of feature decoding may be stored in results database 296, or in any other suitable data repository.

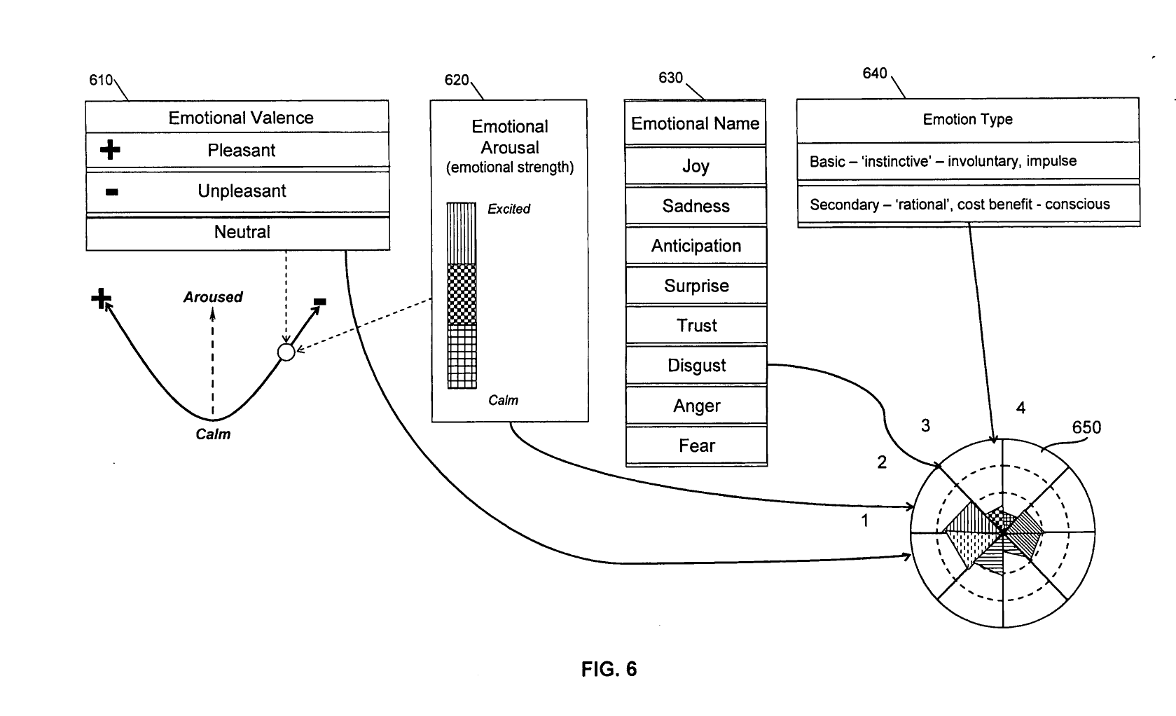

As depicted in the block diagram of FIG. 6, examples of emotional components may include emotional valence 610, emotional arousal 620, emotion category (or name) 630, and/or emotion type 640. Other components may also be determined. As illustrated, emotional valence 610 may be used to indicate whether a user's emotional response to a given stimulus is a positive emotional response (e.g., pleasant or “like”), a negative emotional response (e.g., unpleasant or “dislike”), or a neutral emotional response. Emotional arousal 620 may comprise an indication of the intensity or “emotional strength” of the response. In one implementation, this value may be quantified on a negative to positive scale, with zero indicating a neutral response. Other measurement scales may be implemented.

According to an aspect of the invention, the rules defined in emotional reaction analysis module 224 (FIG. 4) may be based on established scientific findings regarding the study of various eye properties and their meanings. For example, a relationship exists between pupil size and arousal. Additionally, there is a relationship between a user's emotional valence and pupil dilation. An unpleasant or negative reaction, for example, may cause the pupil to dilate larger than a pleasant or neutral reaction.

Blink properties also aid in defining a user's emotional valence and arousal. With regard to valence, an unpleasant response may be manifested in quick, half-closed blinks. A pleasant, positive response, by contrast, may result in long, closed blinks. Negative or undesirable stimuli may result in frequent surprise blinks, while pleasant or positive stimuli may not result in significant surprise blinks. Emotional arousal may be evaluated, for example, by considering the velocity of blinks. Quicker blinks may occur when there is a stronger emotional reaction.

Eye position and movement may also be used to deduce emotional cues. By measuring how long a user fixates on a particular stimulus or portion of a stimulus, a determination can be made as to whether the user's response is positive (e.g., pleasant) or negative (e.g., unpleasant). For example, a user staring at a particular stimulus may indicate a positive (or pleasant) reaction to the stimulus, while a negative (or unpleasant) reaction may be inferred if the user quickly looks away from a stimulus.

As recited above, emotion category (or name) 630 and emotion type 640 may also be determined from the data processed by data processing module 236. Emotion category (or name) 630 may refer to any number of emotions (e.g., joy, sadness, anticipation, surprise, trust, disgust, anger, fear, etc.) described in any known or proprietary emotional model. Emotion type 640 may indicate whether a user's emotional response to a given stimulus is instinctual or rational, as described in greater detail below. Emotional valence 610, emotional arousal 620, emotion category (or name) 630, and/or emotion type 640 may each be processed to generate a map 650 of an emotional response, also described in detail below.

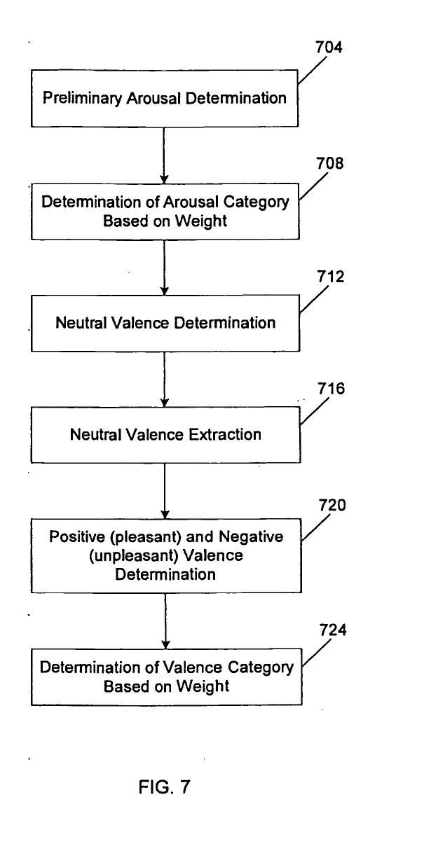

As recited above, one or more rules from emotion reaction analysis module 224 may be applied to the extracted feature data to determine one or more emotional components. Various rules may be applied in various operations. FIG. 7 illustrates a general overview of exemplary feature decoding operations, according to the invention, in one regard. Feature decoding according to FIG. 7 may be performed by emotion reaction analysis module 224. As described in greater detail below, feature decoding may comprise preliminary arousal determination (operation 704), determination of arousal category based on weights (operation 708), neutral valence determination (operation 712) and extraction (operation 716), positive (e.g., pleasant) and negative (e.g., unpleasant) valence determination (operation 720), and determination of valence category based on weights (operation 724). Each of the operations will be discussed in greater detail below along with a description of rules that may be applied in each. For some uses, not all of the operations need be performed. For other uses, additional operations may be performed along with some or all of the operations shown in FIG. 7. In some implementations, one or more operations may be performed simultaneously.

Moreover, the rules applied in each operation are also exemplary, and should not be viewed as limiting. Different rules may be applied in various implementations. As such, the description should be viewed as exemplary, and not limiting.

Prior to presenting the operations and accompanying rules, a listing of features, categories, weights, thresholds, and other variables are provided below.

IAPS Features

Vlevel.IAPS.Value [0;10]
Vlevel.IAPS.SD [0;10]
Alevel.IAPS.Value [0;10]
Alevel.IAPS.SD [0;10]

Variable may be identified according to the International Affective Picture System which characterizes features including a valence level (Vlevel) and arousal level (Alevel). A variable for value and standard deviation (SD) may be defined.

IAPS Categories determined from Features

Vlevel.IAPS.Cat
Alevel.IAPS.Cat

A category variable may be determined from the variables for a valence level and an arousal level. For example, valence level categories may include pleasant and unpleasant. Arousal level categories may be grouped relative to Arousal level I (AI), Arousal level II (AII), and Arousal level III (AIII).

IAPS Thresholds

Vlevel.IAPS.Threshold:
If Vlevel.IAPS.Value <4.3 and Alevel.IAPS.Value >3 then
Vlevel.IAPS.Cat = U
If Vlevel.IAPS.Value > 5.7 and Alevel.IAPS.Value >3 then
Vlevel.IAPS.Cat = P
Else N
Alevel.IAPS.Threshold:
If Alevel.IAPS.Value <3 then Alevel.IAPS.Cat = AI
If Alevel.IAPS.Value >6 then Alevel.IAPS.Cat =AIII
Else N

Predetermined threshold values for feature variables (Vlevel.IAPS.Value, Alevel.IAPS.Value) may be used to determine the valence and arousal category. For example, if a valence level value is less than a predetermined threshold (4.3) and the arousal level value is greater than a predetermined threshold (3) then the valence level category is determined to be unpleasant. Similar determination may be made for an arousal category.

Arousal Features

Alevel.SizeSubsample.Pupil.Size.Mean.MeanLR [0;0.3]
Alevel.Magnitudelntegral.Blink.Count*Length.Frequency(>0).MeanLR
[0;1]

Arousal may be determined from feature values including, but not necessarily limited to, pupil size and/or blink count and frequency.

Arousal Thresholds

Alevel.Size.Subsample.Threshold.AI-AII = 0.1
Alevel.SizeSubsample.Threshold.AII-AIII = 0.15
Alevel.Magnitudelntegral.Threshold.AIII-AII = 0.3
Alevel.Magnitudelntegral.Threshold.AII-AI = 0.45

Predetermined threshold values for arousal features may be used to define the separation between arousal categories (AI, AII, AIII). In this and other examples, other threshold values may be used.

Arousal SD Groups

Alevel.SizeSubsample.Pupil.SD.Group.AI
Alevel.SizeSubsample.Pupil.SD.Group.AII
Alevel.SizeSubsample.Pupil.SD.Group.AIII
Alevel.Magnitudelntegral.Blink.SD.Group.AI
Alevel.Magnitudelntegral.Blink.SD.Group.AII
Alevel.Magnitudelntegral.Blink.SD.Group.AIII

Variables for standard deviation within each arousal category based on arousal features may be defined.

Arousal SDs, Categories and Weights determined from Features

Alevel.SizeSubsample.Pupil.SD
Alevel.SizeSubsample.Pupil.Cat
Alevel.SizeSubsample.Pupil.Cat.Weight
Alevel.MagnitudeIntegral.Blink.SD
Alevel.MagnitudeIntegral.Blink.Cat
Alevel.MagnitudeIntegral.Blink.Cat.Weight

Variables for arousal standard deviation, category and weight for each arousal features may further be defined.

Valence Features

Vlevel.TimeBasedist.Pupil.tBase>>>>2000ms.Mean.MeanLR [0;1800]
Vlevel.BaseIntegral.Pupil.tBase>>>>tAmin.Median.MeanLR [0;1]
Vlevel.Frequency.Blink.Count.Mean.MeanLR [1;3]
Vlevel.PotentionIntegral.Blink.1/DistNextBlink.Mean.MeanLR [0;0.5]
Vlevel.TimeAmin.Pupil.Amin.Median5Mean10.ClusterLR [0;1800]

Valence may be determined from feature values including, but not necessarily limited to, pupil and/or blink data.

Valence Thresholds

Vlevel.TimeBasedist.Threshold.N = (0),
Vlevel.TimeBasedist.Threshold.U-P = 950
Vlevel.BaseIntegral.Threshold.U-P = 0.17
Vlevel.Frequency.Threshold.P-U = 1.10
Vlevel.PotentionIntegral.Threshold.P-U = 0.24
Vlevel.TimeAmin.Threshold.U-P = 660
Vlevel.Neutral.Weight.Threshold = 0.60

Predetermined threshold values for valence features may be used to define the separation between valence categories (pleasant and unpleasant). In this and other examples, other threshold values may be used.

Valence SD Groups

Vlevel.BaseIntegral.Pupil.SD.Group.U
Vlevel.BaseIntegral.Pupil.SD.Group.P
Vlevel.Frequency.Blink.SD.Group U
Vlevel.Frequency.Blink.SD.Group.P
Vlevel.PotentionIntegral.Blink.SD.Group.U
Vlevel.PotentionIntegral.Blink.SD.Group.P
Vlevel.TimeAmin.Pupil.SD.Group.U
Vlevel.TimeAmin.Pupil.SD.Group.P

Variables for standard deviation within each valence category based on valence features may be defined.

Valence SDs, Categories and Weights determined from Features

Vlevel.TimeBasedist.Pupil.SD
Vlevel.TimeBasedist.Pupil.Cat
Vlevel.TimeBasedist.Pupil.Weight
Vlevel.BaseIntegral.Pupil.SD
Vlevel.BaseIntegral.Pupil.Cat
Vlevel.BaseIntegral.Pupil.Weight
Vlevel.Frequency.Blink.SD
Vlevel.Frequency.Blink.Cat
Vlevel.Frequency.Blink.Weight
Vlevel.PotentionIntegral.Blink.SD
Vlevel.PotentionIntegral.Blink.Cat
Vlevel.PotentionIntegral.Blink.Weight
Vlevel.TimeAmin.Pupil.SD
Vlevel.TimeAmin.Pupil.Cat
Vlevel.TimeAmin.Pupil.Weight
Vlevel.Alevel.Cat
Vlevel.Alevel.Weight

Variables for valence standard deviation, category and weight for each valence features may further be defined.

Final Classification and Sureness of correct hit determined from Features

Vlevel.EmotionTool.Cat
Vlevel.Bullseye.Emotiontool.0-100%(Weight)
Alevel.EmotionTool.Cat
Alevel.Bullseye.Emotiontool.0-100%(Weight)
Vlevel.IAPS.Cat
Vlevel.Bullseye.IAPS.0-100%
Alevel.IAPS.Cat
Alevel.Bullseye.IAPS.0-100%

One or more of the foregoing variables reference “IAPS” (or International Affective Picture System) as known and understood by those having skill in the art. In the exemplary set of feature decoding rules described herein, IAPS data is used only as a metric by which to measure basic system accuracy. It should be recognized, however, that the feature decoding rules described herein are not dependent on IAPS, and that other accuracy metrics (e.g., GSR feedback data) may be used in place of, or in addition to, IAPS data.

In one implementation, operation 704 may comprise a preliminary arousal determination for one or more features. Arousal, as described above, comprises an indication of the intensity or “emotional strength” of a response. Each feature of interest may be categorized and weighted in operation 704 and preliminary arousal levels may be determined, using the rules set forth below.

Features used to determine preliminary arousal include:

Alevel.SizeSubsample.Pupil.Size.Mean.MeanLR
Alevel.MagnitudeIntegral.Blink.Count*Length.Frequency(>0).MeanLR
Alevel.BaseIntegral.Pupil.tAmin>>>>tBasedist.Median.MeanLR
used to preliminarily determine Arousal level; AI, AII, AIII.

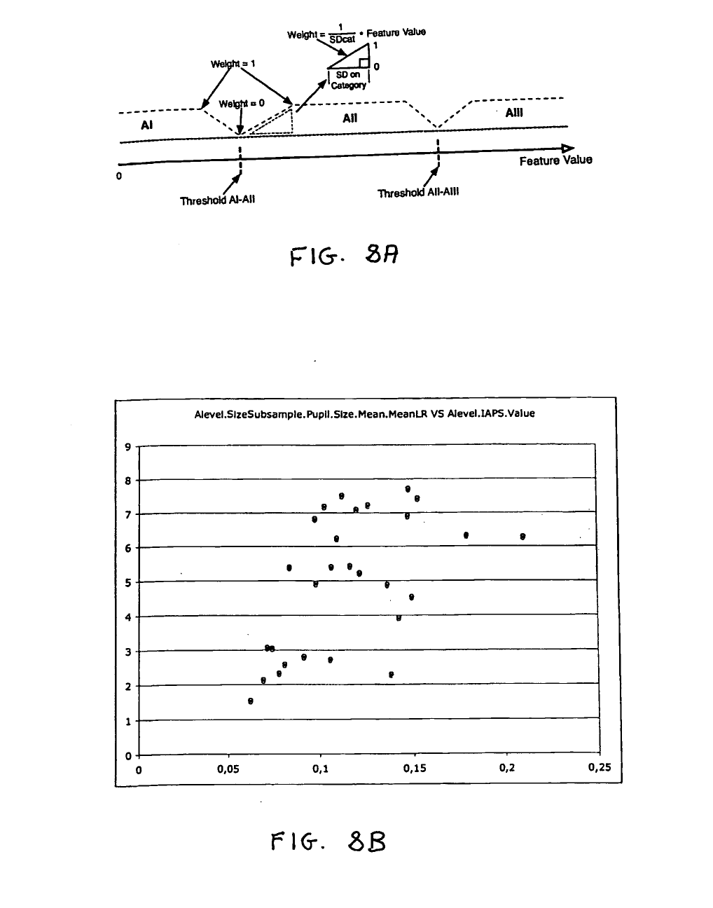

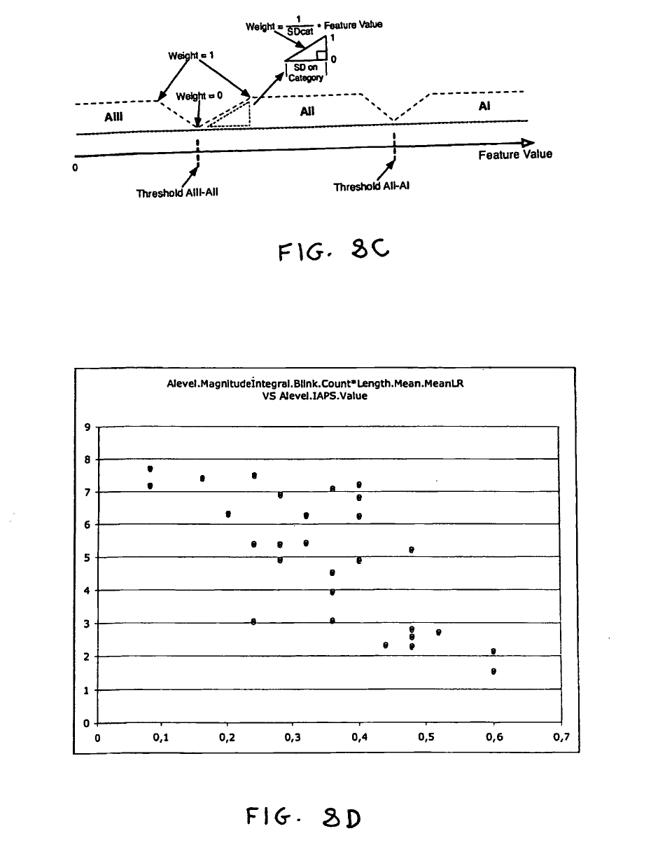

Each feature may be categorized (AI, AII, or AIII) and then weighted according to the standard deviation (SD) for the current feature and category between zero and one to indicate confidence on the categorization. FIG. 8A is a schematic depiction illustrating the determination of Alevel.SizeSubsample.Pupil.Cat and Weight. As shown, the three arousal categories may be defined using threshold values. A weight within each category may be determined according to a feature value divided by the standard deviation for the current feature. Below are a set of iterations used to determine the category and weight based on the arousal feature related to pupil size (Alevel.SizeSubsample.Pupil.Size.Mean.MeanLR).

Determine Alevel.SizeSubsample.Pupil.Cat and Weight

```
If Alevel.SizeSubsample.Pupil.Size.Mean.MeanLR
<Alevel.SizeSubsample.Threshold.AI-AII
then Alevel.SizeSubsample.Pupil.Cat = AI
    If Alevel.SizeSubsample.Pupil.Size.Mean.MeanLR<
    (Alevel.SizeSubsample.Threshold.AI-AII −
    Alevel.SizeSubsample.Pupil.SD.GroupAI)
    then Alevel.SizeSubsample.Pupil.Cat.Weight = 1
    Else Alevel.SizeSubsample.Pupil.Cat.Weight = (1/
    Alevel.SizeSubsample.Pupil.SD.Group.AI)*
    (Alevel.SizeSubsample.Threshold.AI-AII −
    Alevel.SizeSubsample.Pupil.Size.Mean.MeanLR)
```

This part of the iteration determines whether the value for pupil size is less than a threshold value for pupil size between AI and AII. If so, then the category is AI. This part of the iteration goes on to determine the value of the weight between zero and one.

```
If Alevel.SizeSubsample.Pupil.Size.Mean.MeanLR >
Alevel.SizeSubsample.Threshold.AII-AIII
then Alevel.SizeSubsample.Pupil.Cat = AIII
    If Alevel.SizeSubsample.Pupil.Size.Mean.MeanLR >
    (Alevel.SizeSubsample.Threshold AII-AIII +
    Alevel.SizeSubsample.Pupil.SD.Group.AIII)
    then Alevel.SizeSubsample.Pupil.Cat.Weight = 1
    Else Alevel.SizeSubsample.Pupil.Cat.Weight = (1/
    Alevel.SizeSubsample.Pupil.SD.Group.AIII)*
    (Alevel.SizeSubsample.Pupil.Size.Mean.MeanLR −
    Alevel.SizeSubsample.Threshold.AII-AIII)
```

This part of the iteration determines whether the value for pupil size is greater than a threshold value for pupil size between AII and AIII. If so, then the category is AIII. This iteration goes on to determine the value of the weight between zero and one.

```
Else Alevel.SizeSubsample.Pupil.Cat = AII
    If Alevel.SizeSubsample.Pupil.Size.Mean.MeanLR >
    (Alevel.SizeSubsample.Threshold.AI-AII +
    Alevel.SizeSubsample.Pupil.SD.Group.AII) and
    Alevel.SizeSubsample.Pupil.Size.Mean.MeanLR <
    (Alevel.SizeSubsample.Threshold.AII-AIII −
    Alevel.SizeSubsample.Pupil.SD.Group.AII)
    then Alevel.SizeSubsample.Pupil.Cat.Weight = 1
    Else If Alevel.SizeSubsample.Pupil.Size.Mean.MeanLR <
    (Alevel.SizeSubsample.Threshold.AI-AII +
    Alevel.SizeSubsample.Pupil.SD.Group.AII)
    then Alevel.SizeSubsample.Pupil.Cat.Weight = (1/
    Alevel.SizeSubsample.Pupil.SD.Group.AII)*
    (AIevel.SizeSubsample.Pupil.Size. Mean.MeanLR −
    Alevel.SizeSubsample.Threshold.AI-AII)
    else Alevel.SizeSubsample.Pupil.Cat.Weight = (1/
    Alevel.SizeSubsample.Pupil.SD.Group.AII)*
    (Alevel.SizeSubsample.Threshold.AII-AIII −
    Alevel.SizeSubsample.Pupil.Size.Mean.MeanLR)
```

This part of the iteration determines that the category is AII, based on failure to fulfill the proceeding If statements. The iteration goes on to determine the value of the weight between zero and one.

FIG. 8B depicts a plot of Alevel.SizeSubsample.Pupil.Size.Mean.MeanLR versus Alevel.IAPS.Value. The plot values are visually represented in FIG. 8B. FIG. 8C is a schematic depiction illustrating the determination of Alevel.MagnitudeIntegral.Blink.Cat and Weight. Similar to FIG. 8A, the three arousal categories may be defined using threshold values. A weight within each category may be determined according to a feature value divided by the standard deviation for the current feature. Below are a set of iterations used to determine the category and weight based on the arousal feature related to blink data (Alevel.MagnitudeIntegral.Blink.Cat).

Determine Alevel.MagnitudeIntegral.Blink.Cat and Weight

```
If
Alevel.MagnitudeIntegral.Blink.Count*Length.-
Frequency(>0).MeanLR<
Alevel.MagnitudeIntegral.Threshold.AIII-AII then
Alevel.MagnitudeIntegral.Blink.Cat=AIII
    If Alevel.MagnitudeIntegral.Blink.Count*Length.Frequency
    (>0).MeanLR<(Alevel.MagnitudeIntegral.Threshold.AIII-AII−
    Alevel.MagnitudeIntegral.Blink.SD.Group.AIII) then
    Alevel.MagnitudeIntegral.Blink.Cat.Weight=1
    Else Alevel.MagnitudeIntegral.Blink.Cat.Weight = (1/
    Alevel.MagnitudeIntegral.Blink.SD.Group.AIII)*
    Alevel.MagnitudeIntegral.Threshold.AIII-AII −
    Alevel.MagnitudeIntegral.Blink.Count*Length.-
    Frequency(>0).MeanLR)
```

This part of the iteration determines whether the value for blink data is less than a threshold value for the blink data between AIII and AII (also shown in FIG. 8C). If so, then the category is AIII. The part of the iteration goes on to determine the value of the weight between zero and one.

```
If Alevel.MagnitudeIntegral.Blink.Count*Length.-
Frequency(>0).MeanLR >
Alevel.MagnitudeIntegral.Threshold.AII-AI
then Alevel.MagnitudeIntegral.Blink.Cat = AI
    If Alevel.MagnitudeIntegral.Blink.Count*Length.-
    Frequency(>0).MeanLR
    > (Alevel.MagnitudeIntegral.Threshold.AII-AI +
    Alevel.MagnitudeIntegral.Blink.SD.Group.AI)
    then Alevel.MagnitudeIntegral.Blink.Cat.Weight = 1
    Else Alevel.MagnitudeIntegral.Blink.Cat.Weight = (1/
    Alevel.MagnitudeIntegral.Blink.SD.Group.AI)*
    (Alevel.MagnitudeIntegral.Blink.Count*Length.-
    Frequency(>0).MeanLR −
    Alevel.MagnitudeIntegral.Threshold.AII-AI)
```

This part of the iteration determines whether the value for blink data is greater than a threshold value for blink data between AII and AI. If so, then the category is AI. This part of the iteration goes on to determine the value of the weight between zero and one.

```
Else Alevel.MagnitudeIntegral.Blink.Cat = AII
    If
    Alevel.MagnitudeIntegral.Blink.Count*Length.-
    Frequency(>0).MeanLR>
    (Alevel.MagnitudeIntegral.Threshold.AIII-AII +
    Alevel.MagnitudeIntegral.Blink.SD.Group.AII) and
    Alevel.MagnitudeIntegral.Blink.Count*Length.-
    Frequency(>0).MeanLR <
    (Alevel.MagnitudeIntegral.Threshold.AII-AI −
    Alevel.MagnitudeIntegral.Blink.SD.Group.AII)
    then Alevel.MagnitudeIntegral.Blink.Cat.Weight = 1
    Else if
    Alevel.MagnitudeIntegral.Blink.Count*Length.-
    Frequency(>0).MeanLR <
    (Alevel.MagnitudeIntegral.Threshold.AIII-AII +
    Alevel.MagnitudeIntegral.Blink.SD.Group.AII) then
    Alevel.MagnitudeIntegral.Blink.Cat.Weight = (1/
    Alevel.MagnitudeIntegral.Blink.SD.Group.AII)*
    (Alevel.MagnitudeIntegral.Blink.Count*Length.-
    Frequency(>0).MeanLR −
    Alevel.MagnitudeIntegral.Threshold.AIII-AII) else
    Alevel.MagnitudeIntegral.Blink.Cat.Weight = (1/
    Alevel.MagnitudeIntegral.Blink.SD.Group.AII)*
    (Alevel.MagnitudeIntegral.Threshold.AII-AI −
    Alevel.MagnitudeIntegral.Blink.Count*Length.-
    Frequency(>0).MeanLR)
```

This part of the iteration determines that the category is All, based on failure to fulfill the proceeding If statements. The iteration goes on to determine the value of the weight between zero and one.

FIG. 8D depicts a plot of Alevel.MagnitudeIntegral.Blink.Count *Length.Mean.MeanLR versus Alevel.IAPS.Value.

Operation 708 may include the determination of an arousal category (or categories) based on weights. In one implmentation, Alevel.EmotionTool.Cat {AI;AII;AIII} may be determined by finding the Arousal feature with the highest weight. Alevel.EmotionTool.Cat=Max(Sum Weights AI, Sum WeightsAII, Sum Weights AIII).Cat

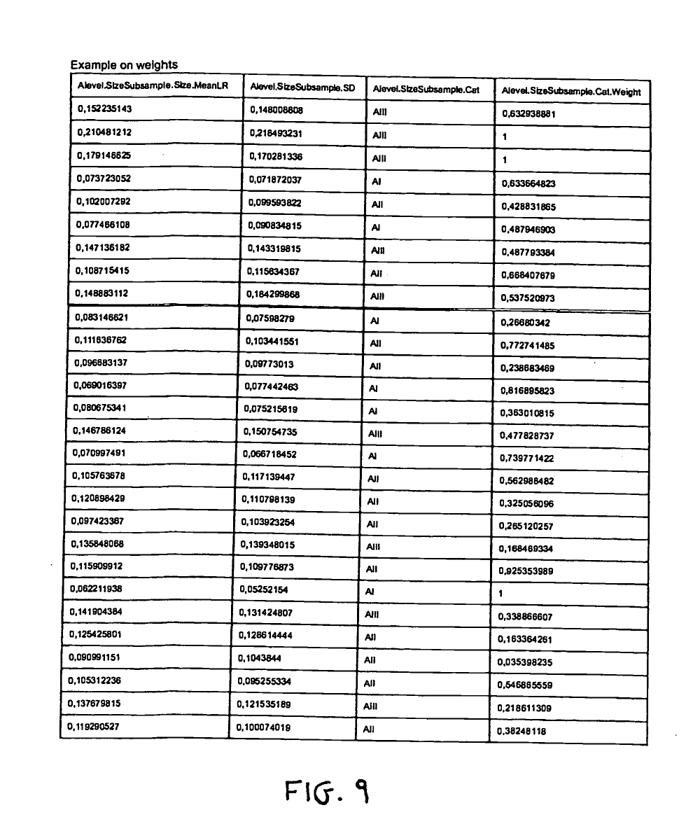

FIG. 9 depicts a table including the following columns:.
(1) Alevel.SizeSubsample.Size.MeanLR;
(2) Alevel.SizeSubsample.SD;
(3) Alevel.SizeSubsample.Cat; and
(4) Alevel.SizeSubsample.Cat.Weight

As recited above, emotional valance may be used to indicate whether a user's emotional response to a given stimulus is a positive emotional response (e.g., pleasant), a negative emotional response (e.g., unpleasant), or a neutral emotional response. In operation 712, rules may be applied for neutral valence determination (to determine if a stimulus is neutral or not).

```
Features used to determine neutral valence:
    Vlevel.TimeBasedist.Pupil.tBase>>>>2000ms.Mean.MeanLR
    Vlevel.TimeAmin.Pupil.Amin.Median5Mean10.ClusterLR
And arousal determination
    Alevel.EmotionTool.Cat
Is used to determine whether a stimulus is Neutral.
    If Vlevel.TimeBasedist.Pupil.tBase>>>>2000ms.Mean.MeanLR = 0 and
    Vlevel.Frequency.Blink.Count.Mean.MeanLR ≧1.25
    then Vlevel.TimeBasedist.Pupil.Cat = Neutral and
    Vlevel.TimeBasedist.Pupil.Weight = 0.75
    If Vlevel.TimeBasedist.Pupil.tBase>>>>2000ms.Mean.MeanLR = 0 and
    Alevel.EmotionTool.Cat = AI then Vlevel.TimeBasedist.Pupil.Cat = Neutral and
    Vlevel.TimeBasedistPupil.Weight = 0.75
    If Alevel.EmotionTool.Cat = AI
    then Vlevel.TimeBasedist.Pupil.Cat = Neutral and
    Vlevel.TimeBasedist.Pupil.Weight = 0.75
    If Vlevel.TimeAmin.Pupil.Amin.Median5Mean10.ClusterLR ≧1000
    thenVlevel.TimeAmin.Pupil.Cat = Neutral and Vlevel.TimeAmin.Pupil.Weight =
    0.50
    Else If Vlevel.TimeAmin.Pupil.Amin Median5Mean10.ClusterLR ≧1300
    then Vlevel.TimeAmin.Pupil.Cat = Neutral and Vlevel.TimeAmin.Pupil.Weight
    = 1.00
```

Four cases may be evaluated:
(1) If the basedistance is zero and the Blink Frequency is greater than 1.25, the response may be considered neutral.
(2) If the basedistance is zero and the Arousal Category is AI, the response may be considered neutral.
(3) If the basedistance is zero and the Arousal Minimum Time is greater than 1000, the response may be considered neutral.
(4) If the Arousal Category is AI, the response may be considered neutral.

In an operation 716, stimulus determined as neutral may be excluded from stimulus evaluation also known as neutral valence extraction.

Exclude stimulus determined as Neutral with weight>Vlevel.Neutral.Weight.Threshold.

If (Vlevel.TimeBasedist.Pupil.Weight +
Vlevel.TimeAmin.Pupil.Weight) >
Vlevel.Neutral.Weight then (if not set above)

Vlevel.TimeBasedist.Pupil.Cat = Neutral
Vlevel.TimeBasedist.Pupil.Weight = 0
Vlevel.TimeAmin.Pupil.Cat = Neutral
Vlevel.TimeAmin.Pupil.Weight = 0
Vlevel.BaseIntegral.Pupil.Cat = Neutral
Vlevel.BaseIntegral.Pupil.Weight = 0
Vlevel.Frequency.Blink.Cat = Neutral
Vlevel.Frequency,Blink.Weight = 0
Vlevel.PotentionIntegral.Blink.Cat = Neutral
Vlevel.PotentionIntegral.Blink.Weight = 0

In operation 720, a determination may be made as to whether a stimulus is positive (e.g., pleasant) or negative (e.g., unpleasant).

Features used to determine pleasant and unpleasant valence include:

Vlevel.TimeBasedist.Pupil.tBase>>>>2000ms.Mean.MeanLR
Vlevel.BaseIntegral.Pupil.tBase>>>>tAmin.Median.MeanLR
Vlevel.Frequency.Blink.Count.Mean.MeanLR
Vlevel.PotentionIntegral.Blink.1/DistNextBlink. Mean.MeanLR
Vlevel.TimeAmin.Pupil.Amin.Median5Mean10.ClusterLR

These features are used to determine if stimulus is Pleasant or Unpleasant.

All or selected features can be categorized and then weighted according to the standard deviation for the current feature and category between zero and one to indicate confidence on the categorization.

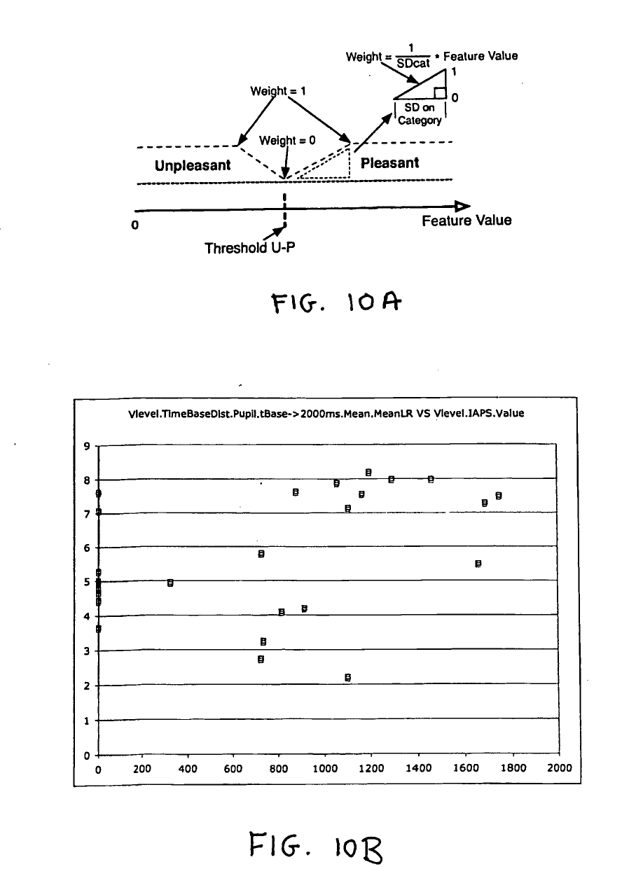

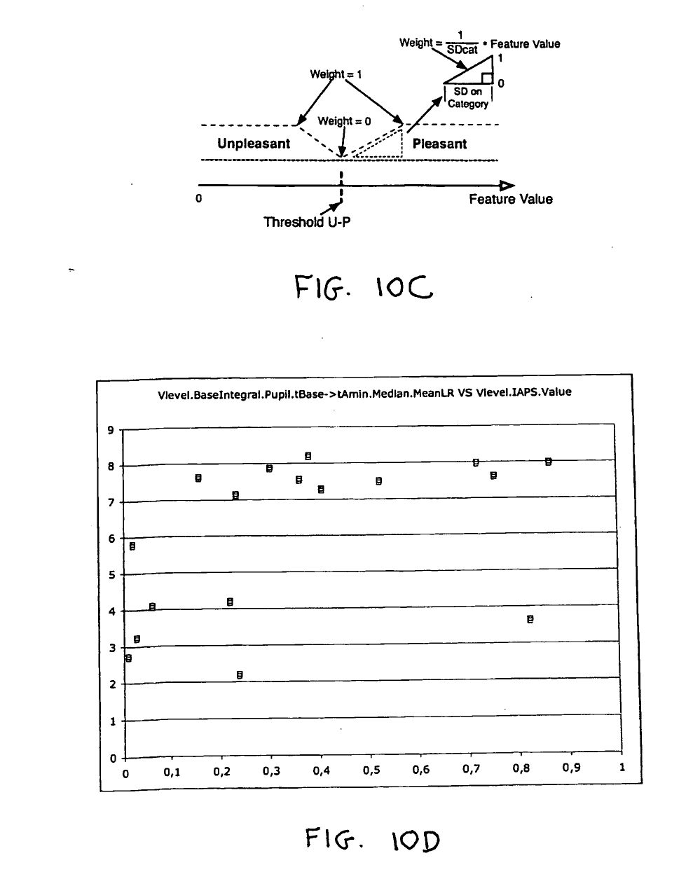

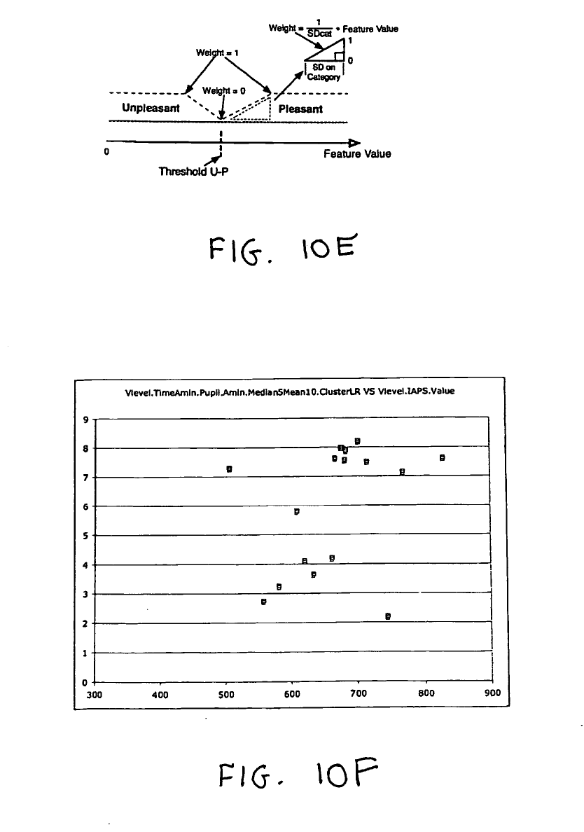

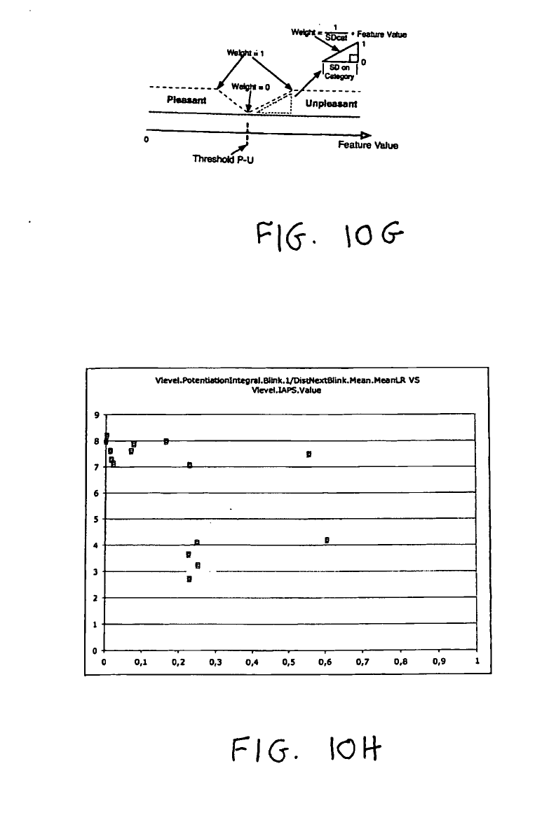

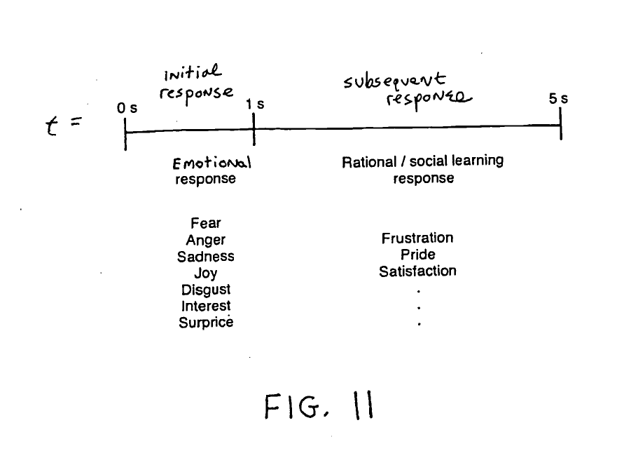

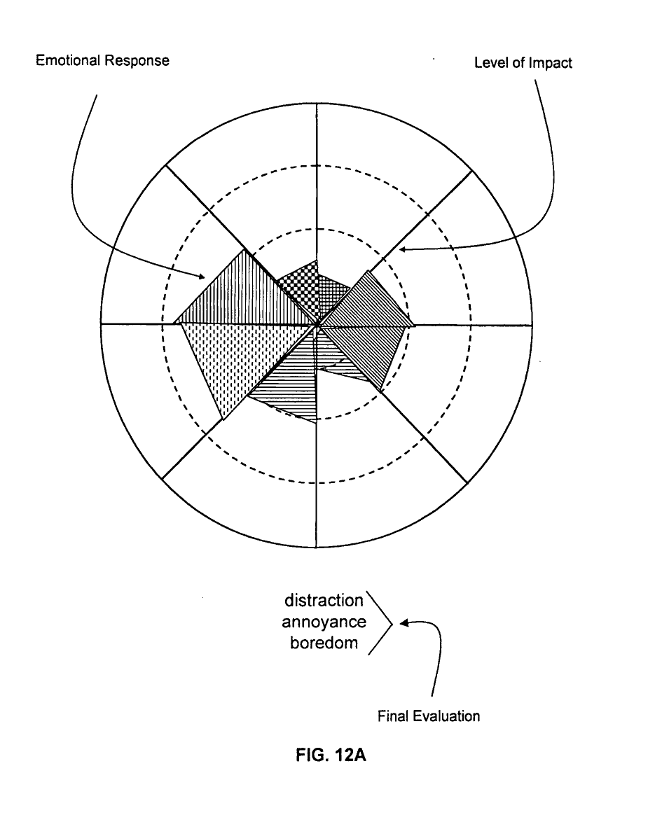

FIG. 10A is a schematic depiction illustrating the determination of Vlevel.TimeBasedist.Pupil.Cat and Weight. As shown, the two valence categories may be defined using threshold values. A weight within each category may be determined according to a feature value divided by the standard deviation for the current feature. Below are a set of iterations used to determine the category and weight based on the valence feature related to pupil data (Vlevel.TimeBasedist.Pupil.tBase>>>>2000ms.Mean.MeanLR).

Determine Vlevel.TimeBasedist.Pupil.Cat and Weight

```
If Vlevel.TimeBasedist.Pupil.Cat ≠Neutral then
    If Vlevel.TimeBasedist.Pupil.tBase>>>>2000ms.Mean.MeanLR <
    Vlevel.TimeBasedist.Threshold.U-P then
    Vlevel.TimeBasedistPupil.Cat = Unpleasant
```

If Vlevel.TimeBasedist.Pupil.tBase>>>>2000ms.Mean.MeanLR <
(Vlevel.TimeBasedist.Threshold.U-P −
Vlevel.TimeBasedist.Pupil.SD.Group.U)
then Vlevel.TimeBasedist.Pupil.Weight = 1
Else Vlevel.TimeBasedist.Pupil.Weight = (1/
Vlevel.TimeBasedist.Pupil.SD.Group.U)*
(Vlevel.TimeBasedist.Threshold.U-P −
Vlevel.TimeBasedist.Pupil.tBase>>>>2000ms.Mean.MeanLR)

Else Vlevel.TimeBasedist.Pupil.Cat = Pleasant

If Vlevel.TimeBasedist.Pupil.tBase>>>>2000ms.Mean.MeanLR >
(Vlevel.TimeBasedist.Threshold.U-P +
Vlevel.TimeBasedist.Pupil.SD.Group.P) then
Vlevel.TimeBasedist.Pupil.Weight = 1
Else Vlevel.TimeBasedist.Pupil.Weight = (1/
Vlevel.TimeBasedistPupil.SD.Group.P)*
(Vlevel.TimeBasedist.Pupil.tBase>>>>2000ms.Mean.MeanLR −
Vlevel.TimeBasedist.Threshold.U-P)

Two cases may be evaluated:

(1) If the Basedistance is lower than the TimeBasedist.Threshold, then the response may be considered unpleasant.

(2) If the Basedistance is greater than the TimeBasedist.Threshold then, then the reponse may be considered pleasant.

FIG. 10B depicts a plot of Vlevel.TimeBasedist.Pupil.tbase->2000ms.Mean.MeanLR versus Vlevel.IAPS.Value.

FIG. 10C is a schematic depiction illustrating the determination of Vlevel.BaseIntegral.Pupil.Cat and Weight. As shown, the two valence categories may be defined using threshold values. A weight within each category may be determined according to a feature value divided by the standard deviation for the current feature. Below are a set of iterations used to determine the category and weight based on the valence feature related to pupil data (Vlevel.TimeBasedist.Pupil.tBase>>>>tAmin.Mean.MeanLR).

Determine Vlevel.BaseIntegral.Pupil.Cat and Weight

```
If Vlevel.BaseIntegral.Pupil.Cat ≠Neutral then
    If Vlevel.BaseIntegral.Pupil.tBase>>>>tAmin.Median.MeanLR <
    Vlevel.BaseIntegral.Threshold.P-U
    then Vlevel.BaseIntegral.Pupil.Cat = Unpleasant
```

If Vlevel.BaseIntegral.Pupil.tBase>>>>tAmin.Median.MeanLR <
(Vlevel.BaseIntegral.Threshold.P-U −
Vlevel.BaseIntegral.Pupil.SD.Group.U)
then Vlevel.BaseIntegral.Pupil.Weight = 1
Else Vlevel.BaseIntegral.Pupil.Weight = (1/
Vlevel.BaseIntegral.Pupil.SD.Group.U)*
(Vlevel.BaseIntegral.Threshold.P-U −
Vlevel.BaseIntegral.Pupil.tBase>>>>tAmin.Median.MeanLR)

Else Vlevel.BaseIntegral.Pupil.Cat = Pleasant

If Vlevel.BaseIntegral.Pupil.tBase>>>>tAmin.Median.MeanLR >
(Vlevel.BaseIntegral.Threshold.P-U +
Vlevel.BaseIntegral.Pupil.SD.Group.P)
then Vlevel.BaseaIntegral.Pupil.Weight = 1
Else Vlevel.BaseIntegral.Pupil.Weight = (1/
Vlevel.BaseIntegral.Pupil.SD.Group.P)*
(Vlevel.BaseIntegral.Pupil.tBase>>>>tAmin.Median.MeanLR −
Vlevel.BaseIntegral.Threshold.P-U)

Two cases may be evaluated:

(1) If the BaseIntegral is lower than the BaseIntegral.Threshold, then the response may be considered unpleasant.

(2) If the BaseIntegral is greater than the BaseIntegral.Threshold, then the response may be considered pleasant.

FIG. 10D depicts a plot of Vlevel.BaseIntegral.Pupil.tBase->tAmin.Median.MeanLR versus Vlevel.IAPS Value.

FIG. 10E is a schematic depiction illustrating the determination of Vlevel.TimeAminPupil.Cat and Weight. As shown, the two valence categories may be defined using threshold values. A weight within each category may be determined according to a feature value divided by the standard deviation for the current feature. Below are a set of iterations used to determine the category and weight based on the valence feature related to pupil data (Vlevel.TimeAmin.Pupil.Amin.Median5Mean10.ClusterLR).

Determine Vlevel.TimeAminPupil.Cat and Weight

If Vlevel.TimeAmin.Pupil.Cat ≠Neutral then

If Vlevel.TimeAmin.Pupil.Amin.Median5Mean10.ClusterLR <
Vlevel.TimeAmin.Threshold.P-U
then Vlevel.TimeAmin.Pupil.Cat = Unpleasant
If Vlevel.TimeAmin.Pupil.Amin.Median5Mean10.ClusterLR <
(Vlevel.TimeAmin.Threshold.P-U −
Vlevel.TimeAmin.Pupil.SD.Group.U)
then Vlevel.TimeAmin.Pupil.Weight = 1
Else Vlevel.TimeAminPupil.Weight = (1/
Vlevel.TimeAmin.Pupil.SD.Group.U)*
(Vlevel.TimeAmin.Threshold.P-U −
Vlevel.TimeAmin.Pupil.Amin.Median5Mean10.ClusterLR)

Else Vlevel.TimeAmin.Pupil.Cat = Pleasant

If Vlevel.TimeAmin.Pupil.Amin.Median5Mean10.ClusterLR >
(Vlevel.TimeAmin.Threshold.P-U +
Vlevel.TimeAmin.Pupil.SD.Group.P)
then Vlevel.TimeAmin.Pupil.Weight = 1
Else Vlevel.TimeAmin.Pupil.Weight = (1/
Vlevel.TimeAmin.Pupil.SD.Group.P)*
(Vlevel.TimeAmin.Pupil.Amin.Median5.Mean10.ClusterLR −
Vlevel.TimeAmin.Threshold.P-U)

Two cases may be evaluated:

(1) If the arousal minimum time is lower than the arousal minimum time threshold, then the response may be considered unpleasant.

(2) If the arousal minimum time is lower than the arousal minimum time threshold, then the response may be considered pleasant.

FIG. 10F depicts a plot of Vlevel.TimeAmin.Pupil.Amin.Median5Mean10.ClusterLR versus Vlevel.IAPS.Value.

FIG. 10G is a schematic depiction illustrating the determination of Vlevel.PotentionIntegral.Blink and Weight. As shown, the two valence categories may be defined using threshold values. A weight within each category may be determined according to a feature value divided by the standard deviation for the current feature. Below are a set of iterations used to determine the category and weight based on the valence feature related to pupil data (Vlevel.PotentionIntegral.Blink.1/DistNextBlink.Mean.MeanLR).

Determine Vlevel.PotentionIntegral.Blink and Weight

```
If Vlevel.PotentionIntegral.Blink.Cat ≠Neutral then
    If Vlevel.PotentionIntegral.Blink.1/DistNextBlink.Mean.MeanLR <
    Vlevel.PotentionIntegral.Threshold.P-U
    then Vlevel.PotentionIntegral.Blink.Cat = Pleasant
```

If Vlevel.PotentionIntegral.Blink.1/DistNextBlink.Mean.MeanLR
< (Vlevel.PotentionIntegral.Threshold.P-U −
Vlevel.PotentionIntegral.Blink.SD.Group.P)
then Vlevel.PotentionIntegral.Blink.Weight = 1
Else Vlevel.PotentionIntegral.Blink.Weight =
(1/Vlevel.PotentionIntegral.Blink.SD.Group.P)*
(Vlevel.PotentionIntegral.Threshold.P-U −
Vlevel.PotentionIntegral.Blink.Amin.Median5Mean10.ClusterLR)

Else Vlevel.PotentionIntegral.Blink.Cat = Unpleasant

If Vlevel.PotentionIntegral.Blink.1/DistNextBlink.Mean.MeanLR
> (Vlevel.PotentionIntegral.Threshold.P-U +
Vlevel.PotentionIntegral.Blink.SD.Group.U
then Vlevel.PotentionIntegral.Blink.Weight = 1
Else Vlevel.PotentionIntegral.Blink.Weight =
(1/Vlevel.PotentionIntegral.Blink.SD.Group.U)*
(Vlevel.PotentionIntegral.Blink.1 /DistNextBlink.Mean.MeanLR
Vlevel.PotentionIntegral.Threshold.P-U)

Two cases may be evaluated:

(1) If the PotentionIntegral/DistNextBlink is lower than the PotentionIntegral.Threshold, then the response may be considered pleasant.

(2) If the PotentionIntegral/DistNextBlink is greater than the PotentionIntegral.Threshold, then the response may be considered unpleasant.

FIG. 10H depicts a plot of Vlevel.PotentionIntegral.Blink.1/DistNextBlink.Mean.MeanLR versus Vlevel.IAPS.Value.

In an operation 724, a valence category (or categories) maybe determined based on weights:

Determination of Vlevel.EmotionTool.Cat {U;P} by finding the Valence feature with the highest weight.

Vlevel.EmotionTool.Cat=Max(Sum Weights U, Sum Weights P).Cat

A classification table may be provided including the following information:

PRINT TO CLASSIFICATION TABLE ENTRANCES

Stimuli Name
IAPS Rows
Vlevel.IAPS.Value
Vlevel.IAPS.SD
Vlevel.IAPS.Cat
Alevel.IAPS.Value
Alevel.IAPS.SD
Alevel.IAPS.Cat
Arousal Rows
Alevel.SizeSubsampie.Pupil.SIZE.Mean.MeanLR
Alevel.SizeSubsampie.Pupil.SD
Alevel.SizeSubsample.Pupil.Cat
Alevel.SizeSubsample.Pupil.Cat.Weight
Alevel.MagnitudeIntegral.Blink.Count*Length.Frequency(>0).MeanLR
Alevel.MagnitudeIntegral.Blink.SD
Alevel.MagnitudeIntegral.Blink.Cat
Alevel.MagnitudeIntegral.Blink.Cat.Weight
Valence Rows
Vlevel.TimeBasedist.Pupil.tBase>>>>2000ms.Mean.MeanLR
Vlevel.TimeBasedist.Pupil.SD
Vlevel.TimeBasedist.Pupil.Cat
Vlevel.TimeBasedist.Pupil.Weight
Vlevel.BaseIntegral.Pupil.tBase>>>>tAmin.Median.MeanLR
Vlevel.BaseIntegral.Pupil.SD
Vlevel.BaseIntegral.Pupil.Cat
Vlevel.BaseIntegral.Pupil.Weight
Vlevel.Frequency.Blink.Count.Mean.MeanLR
Vlevel.Frequency.Blink.SD
Vlevel.Frequency.Blink.Cat
Vlevel.Frequency.Blink.Weight
Vlevel.PotentionIntegral.Blink.1/DistNextBlink.Mean.MeanLR
Vlevel.PotentionIntegral.Blink.SD
Vlevel.PotentionIntegral.Blink.Cat
Vlevel.PotentionIntegral.Blink.Weight
Vlevel.TimeAmin.Pupil.Amin.Median5Mean10.ClusterLR
Vlevel.TimeAmin.Pupil.SD
Vlevel.TlmeAmin.Pupil.Cat
Vlevel.TlmeAmin.Pupil.Weight
Final Classification Rows
Vlevel.EmotionTool.Cat
Vlevel.Bullseye.EmotionTool.0-100%(Weight)
Alevel.EmotionTool.Cat
Alevel.Bullseye.EmotionTool.0-100%(Weight)
Vlevel.IAPS.Cat
Vlevel.Bullseye.IAPS.0-100%
Vlevel.Hit.Ok
Alevel.IAPS.Cat
Alevel.Bullseye.IAPS.0-100%
Alevel.Hit.Ok

According to another aspect of the invention, a determination may be made as to whether a user has experienced an emotional response to a given stimulus.

In one implementation, processed data may be compared to data collected and processed during calibration to see if any change from the emotionally neutral (or other) state measured (or achieved) during calibration has occurred. In another implementation, the detection of or determination that arousal has been experienced (during the aforementioned feature decoding data processing) may indicate an emotional response.

If it appears that an emotional response has not been experienced, data collection may continue via data collection module 220, or the data collection session may be terminated. By contrast, if it is determined that an emotional response has been experienced, processing may occur to determine whether the emotional response comprises an instinctual or rational-based response.

As illustrated in FIG. 11, within the very first second or seconds of perceiving a stimulus, or upon “first sight,” basic emotions (e.g., fear, anger, sadness, joy, disgust, interest, and surprise) may be observed as a result of activation of the limbic system and more particularly, the amygdala. In many instances, an initial period (e.g., a second) may be enough time for a human being to decide whether he or she likes or dislikes a given stimulus. This initial period is where the emotional impact really is expressed, before the cortex can return the first result of its processing and rational thinking takes over. Secondary emotions such as frustration, pride, and satisfaction, for example, may result from the rational processing of the cortex within a time frame of approximately one to five seconds after perceiving a stimulus. Although there is an active cooperation between the rational and the emotional processing of a given stimulus, it is advantageous to account for the importance of the “first sight” and its indication of human emotions.

According to an aspect of the invention, one or more rules from emotional reaction analysis module 224 may be applied to determine whether the response is instinctual or rational. For example, sudden pupil dilation, smaller blink sizes, and/or other properties may indicate an instinctual response, while a peak in dilation and larger blink sizes may indicate a rational reaction. Other predefined rules may be applied.

If a user's emotional response is determined to be an instinctual response, mapping module 232 (FIG. 4) may apply the data corresponding to the emotional response to an instinctual emotional impact model. If a user's emotional response is determined to be a rational response, mapping module 232 (FIG. 4) may apply the data corresponding to the rational response a rational emotional impact model.

As previously recited, data corresponding to a user's emotional response may be applied to various known emotional models including, but not limited to, the Ekmans, Plutchiks, and Izards models.

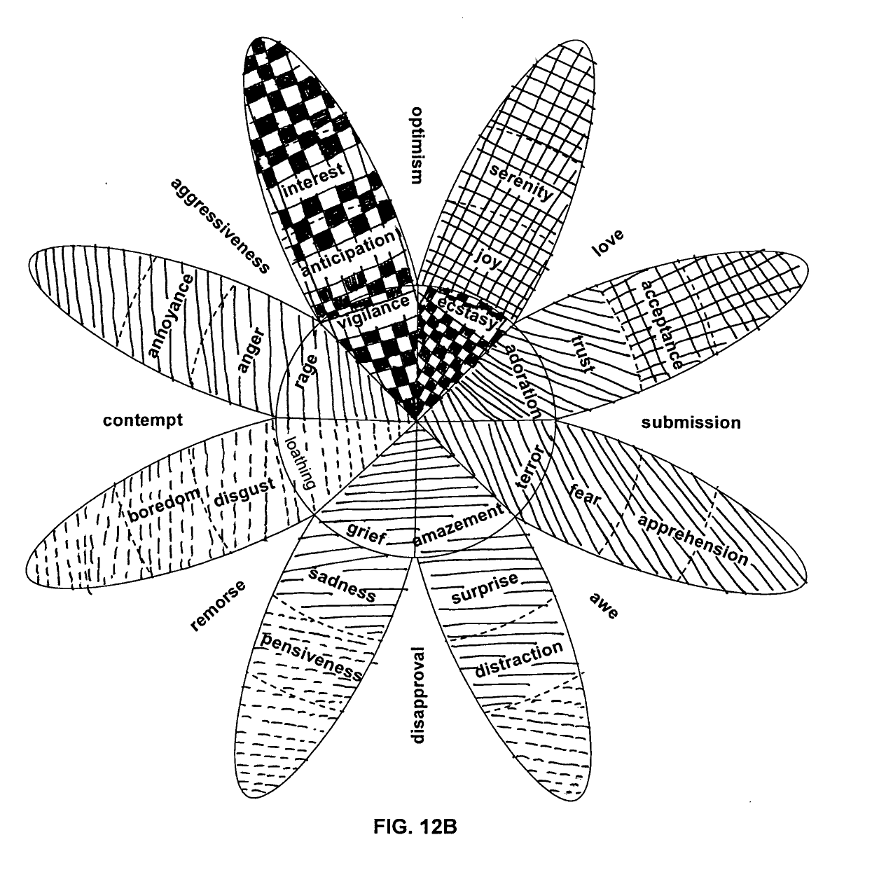

According to an aspect of the invention, instinctual and rational emotional responses may be mapped in a variety of ways by mapping module 232. FIG. 12A is an exemplary illustration of a map of an emotional response, according to one embodiment of the invention. This mapping is based on the Plutchiks emotional model as depicted in FIG. 12B. In one implementation, each emotion category (or name) in a model may be assigned a different color. Other visual indicators may be used. Lines (or makers) extending outward from the center of the map may be used as a scale to measure the level of impact of the emotional response. Other scales may be implemented.

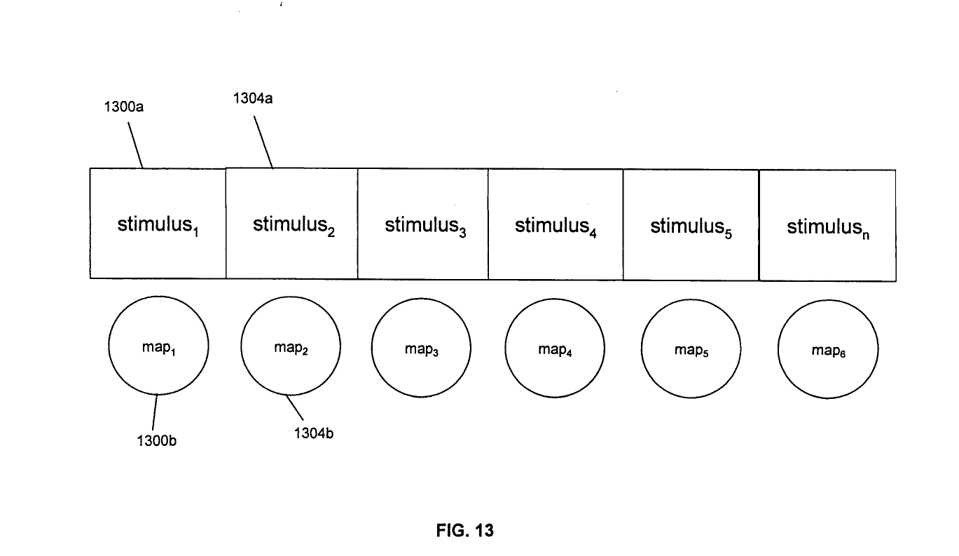

According to an aspect of the invention, these maps may be displayed simultaneously and in synchronization with the stimuli that provoked them. For example, as illustrated in FIG. 13, a first stimulus 1300a may be displayed just above corresponding map 1300b which depicts the emotional response of a user to stimulus 1300a. Similarly, second stimulus 1304a may be displayed just above corresponding map 1304b which depicts the emotional response of a user to stimulus 1304a, and so on. Different display formats may be utilized. In this regard, a valuable analysis tool is provided that may enable, for example, content providers to view all or a portion of a proposed content along with a map of the emotional response it elicits from users.

Collected and processed data may be presented in a variety of manners. According to one aspect of the invention, fro instance, a gaze plot may be generated to highlight (or otherwise illustrate) those areas on a visual stimulus (e.g., a picture) that were the subject of most of a user's gaze fixation while the stimulus was being presented to the user. As previously recited, processing gaze (or eye movement) data may comprise, among other things, determining fixation time (e.g., how long does the eye focus on one point) and the location of the fixation in space as defined by x,y,z or other coordinates. From this information, clusters of fixation points may be identified. In one implementation, a mask may be superimposed over a visual image or stimuli that was presented to a user. Once clusters of fixation points have been determined based on collected and processed gaze data that corresponds to the particular visual stimuli, those portions of the mask that correspond to the determined cluster of fixation points may be made transparent so as to reveal only those portions of the visual stimuli that a user focused on the most. Other data presentation techniques may be implemented.

In one implementation, results may be mapped to an adjective database 298 via a language module (or engine) 240 which may aid in identifying adjectives for a resulting emotional matrix. This may assist in verbalizing or describing results in writing in one or more standardized (or industry-specific) vocabularies.

In yet an alternative implementation, statistics module (or engine) 244 may enable statistical analyses to be performed on results based on the emotional responses of several users or test subjects. Scan-path analysis, background variable analysis, and emotional evaluation analysis are each examples of the various types of statistical analyses that may be performed. Other types of statistical analyses may be performed.

Moreover, in human-machine interactive sessions, the interaction may be enhanced or content may be changed by accounting for user emotions relating to user input and/or other data. The methodology of the invention may be used in various artificial intelligence or knowledge-based systems to enhance or suppress desired human emotions. For example, emotions may be induced by selecting and presenting certain stimuli. Numerous other applications exist.

Depending on the application, emotion detection data (or results) from results database 296 may be published in a variety of manners. Publication may comprise, for example, incorporating data into a report, saving the data to a disk or other known storage device (associated with computer 110), transmitting the data over a network (e.g., the Internet), or otherwise presenting or utilizing the data. The data may be used in any number of applications or in other manners, without limitation.

According to one aspect of the invention, as stimuli is presented to a user, the user may be prompted to respond to command-based inquiries via, for example, keyboard 140, mouse 150, microphone 160, or through other sensory input devices. The command-based inquiries may be verbal, textual, or otherwise. In one embodiment, for example, a particular stimulus (e.g., a picture) may be displayed to a user. After a pre-determined time period, the user may then be instructed to select whether he or she found the stimulus to be positive (e.g., pleasant), negative (e.g., unpleasant), or neutral, and/or the degree. Alternatively, a user may be prompted to respond when he or she has formed an opinion about a particular stimulus or stimuli. The time taken to form an opinion may be stored and used in a variety of ways. Other descriptors may of course be utilized. The user may register selections through any one of a variety of actions or gestures, for example, via a mouse-click in a pop-up window appearing on display device 130, verbally by speaking the response into microphone 160, or by other actions. Known speech and/or voice recognition technology may be implemented for those embodiments when verbal responses are desired. Any number and type of command-based inquiries may be utilized for requesting responses through any number of sensory input devices. Command-based reaction analysis module (or engine) 228 may apply one or more predetermined rules to data relating the user's responses to aid in defining the user's emotional reaction to stimuli. The resulting data may be used to supplement data processed from eye-tracking device 120 to provide enhanced emotional response information.

Other embodiments, uses and advantages of the invention will be apparent to those skilled in the art from consideration of the specification and practice of the invention disclosure herein. Accordingly, the specification should be considered exemplary only.

We claim:

1. A computer implemented method for detecting human emotion in response to presentation of one or more stimuli, based on at least measured physiological data, the method comprising:
presenting at least one stimulus to a subject;
collecting data including physiological data from the subject, the physiological data including pupil data, blink data, and gaze data;
performing eye feature extraction processing to determine eye features of interest from the collected physiological data; and
analyzing the eye features of interest to identify one or more emotional components of a subject's emotional response to the at least one stimulus.

2. The method of claim 1, wherein the method further comprises the step of using the eye features of interest to determine instinctive emotional components of the subject's response to the at least one stimulus.

3. The method of claim 1, wherein the method for analyzing further includes applying rules-based analysis to identify one or more emotional components of the subject's emotional response.

4. The method of claim 1, wherein the step of analyzing further includes applying rules-based analysis to eye features of interest corresponding to the subject's age to identify one or more emotional components of the subject's emotional response.

5. The method of claim 1, wherein the step of analyzing further includes applying rules-based analysis corresponding to the subject's gender to identify one or more emotional components of the subject's emotional response.

6. The method of claim 1, wherein the step of analyzing further includes applying statistical analysis to identify one or more emotional components of subject's emotional response.

7. The method of claim 1, wherein the method further comprises the step of using the eye features of interest to determine rational emotional components of the subject's response to the at least one stimulus.

8. The method of claim 1, wherein the emotional components include emotional valence, emotional arousal, emotion category, and emotion type.

9. The method of claim 1, wherein the method further comprises the step of performing data error detection and correction on the collected physiological data.

10. The method of claim 9, wherein the step of data error detection and correction comprises determination and removal of outlier data.

11. The method of claim 9, wherein the step of data error detection and correction comprises one or more of pupil dilation correction; blink error correction; and gaze error correction.

12. The method of claim 9, wherein the method further comprises the step of storing corrected data and wherein the step of performing eye feature extraction processing is performed on the stored corrected data.

13. The method of claim 1, wherein the method further comprises performing a calibration operation during a calibration mode, the calibration operation including the steps of:
a. calibrating one or more data collection sensors; and
b. determining a baseline emotional level for a subject.

14. The method of claim 13, wherein the step of calibrating one or more data collection sensors includes calibrating to environment ambient conditions.

15. The method of claim 1, wherein the data collection is performed at least in part by an eye-tracking device, and the method further comprises the step of calibrating the eye-tracking device to a subject's eyes prior to data collection.

16. The method of claim 1, further comprising the step of presenting one or more stimuli for inducing, in a subject, a desired emotional state, prior to data collection.

17. The method of claim 1, wherein the step of presenting the at least one stimulus to a subject further comprises presenting a predetermined set of stimuli to a subject and the data collection step comprises separately for each stimulus in the set, the stimulus and the data collected when the stimulus is presented.

18. The method of claim 1 further comprising the step of creating a user profile for a subject to assist in the step of analyzing eye features of interest, wherein the user profile include the subject's eye-related data, demographic information, or calibration information.

19. The method of claim 1, wherein the step of collecting data further comprises collecting environmental data.

20. The method of claim 1, wherein the step of collecting data comprises collecting eye data at a predetermined sampling frequency over a period of time.

21. The method of claim 1, wherein the eye feature data relates to pupil data for pupil size, pupil size change data and pupil velocity of change data.

22. The method of claim 1, wherein the eye feature data relates to pupil data for the time it takes for dilation or contraction to occur in response to a presented stimulus.

23. The method of claim 1 wherein the eye feature data relates to pupil data for pupil size before and after a stimulus is presented to the subject.

24. The method of claim 1, wherein the eye feature data relates to blink data for blink frequency, blink duration, blink potention, and blink magnitude data.

25. The method of claim 1, wherein the eye feature data relates to gaze data for saccades, express saccades and nystagmus data.

26. The method of claim 1, wherein the eye feature data relates to gaze data for fixation time, location of fixation in space, and fixation areas.

27. The method of claim 2, wherein the step of determining the instinctive emotional components further comprises applying a rules-based analysis to the features of interest to determine an instinctual response.

28. The method of claim 2, wherein the step of determining the instinctive emotional components further comprises applying a statistical analysis to the features of interest to determine an instinctual response.

29. The method of claim 1, further comprising the step of mapping emotional components to an emotional model.

30. The method of claim 2, further comprising the step of applying the instinctive emotional components to an instinctive emotional model.

31. The method of claim 7, further comprising the step of applying the rational emotional components to a rational emotional model.

32. The method of claim 1, wherein the method further comprises the step of using the eye features of interest to determine instinctual emotional components and rational emotional components of the subject's response to the at least one stimulus.

33. The method of claim 32, further comprising the step of applying the instinctive emotional components to an instinctive emotional model and applying the rational emotional components to a rational emotional model.

34. The method of claim 1, wherein the method further comprises the step of using the eye features of interest to determine one or more initial emotional components of a subject's emotional response that correspond to an initial period of time that the at least one stimulus is perceived by the subject.

35. The method of claim 34, wherein the method further comprises the step of using the eye features of interest to determine one or more secondary emotional components of a subject's emotional response that correspond to a time period after the initial period of time.

36. The method of claim 34, wherein the method further comprises the step of using the eye features of interest to determine one or more secondary emotional components of a subject's emotional response that correspond to a time period after the initial period of time and further based on the one or more initial emotional components.

37. The method of claim 1, further comprising the step of synchronizing a display of emotional components of the subject's emotional response simultaneously with the corresponding stimulus that provoked the emotional response.

38. The method of claim 1, further comprising the step of synchronizing a time series display of emotional components of the subject's emotional response individually with the corresponding stimulus that provoked the emotional response.

39. The method of claim 1, further comprising the step of applying the emotional components to an emotional adjective database to determine a label for the emotional response based on an emotional response matrix.

40. The method of claim 1, further comprising the step of aggregating for two of more subjects, the emotional response of the subjects to at least one common stimulus.

41. The method of claim 1 further comprising the step of collecting data regarding at least one other physiological property of the subject other than eye data and using the collected data regarding the at least one other physiological property to assist in determining an emotional response of the subject.

42. The method of claim 1 further comprising the step of collecting facial expression data of the subject in response to the presentation of a stimulus and using the collected facial expression data to assist in determining an emotional response of the subject.

43. The method of claim 1 further comprising the step of collecting galvanic skin response data of the subject in response to the presentation of a stimulus and using the collected skin response data to assist in determining an emotional response of the subject.

44. The method of claim 1 wherein the stimuli comprise visual stimuli and at least one non-visual stimulus.

45. The method of claim 29 further comprising the step of outputting the emotional components including whether the subject had a positive emotional response or a negative emotional response, and the magnitude of the emotional response.

46. The method of claim 1 further comprising the step of determining if a subject had a non-neutral emotional response, and if so, outputting an indicator of the emotional response including whether the subject had a positive emotional response or a negative emotional response, and the magnitude of the emotional response.

47. The method of claim 1 further comprising the step of using the one or more identified emotional components of the subject's emotional response as user input in an interactive session.

48. The method of claim 1 further comprising the step of recording in an observational session, the one or more identified emotional components of the subject's emotional response.

49. The method of claim 1 further comprising the step of outputting an indicator of the emotional response including an emotional valence and an emotional arousal, wherein the emotional arousal is represented as a number based on a predetermined numeric scale.

50. The method of claim 1, further comprising the step of outputting an indicator relating to accuracy of an emotional response, wherein the accuracy is presented as a number or a numerical range based on a predetermined numerical scale.

51. The method of claim 1 further comprising the step of outputting an indicator of an emotional response including an instinctive emotional response and a rational emotional response.

52. The method of claim 1 further comprising the step of outputting an indicator of an emotional response including an instinctive emotional response and a secondary emotional response.

53. The method of claim 1 further comprising the step of outputting emotional response maps, where the maps are displayed simultaneously and in juxtaposition with stimuli that caused the emotional response.

54. The method of claim 1, further including the step of prompting the subject to respond to verbal or textual inquiries about a given stimulus while the stimulus is presented to the subject.

55. The method of claim 1 further including the step of prompting the subject to respond to verbal or textual inquiries about a given stimulus after the stimulus has been displayed to the subject for a predetermined time.

56. The method of claim 54, further including the step of recording the time it takes the subject to respond to a prompt.

57. The method of claim 1, wherein the at least one stimulus is a customized stimulus for presentation to the subject for conducting a survey.

58. A computerized system for detecting human emotion in response to presentation of one or more stimuli, based on at least measured physiological data, the system including:
a stimulus module for presenting at least one stimulus to a subject;
a data collection means for collecting data including physiological data from the subject, the physiological data including pupil data, blink data, and gaze data;
a data processing module for performing eye feature extraction processing to determine eye features of interest from the collected physiological data; and
an emotional response analysis module for analyzing the eye features of interest to identify one or more emotional components of a subject's emotional response.
